(19) United States
(12) Patent Application Publication
(10) Pub. No.: US 2009/0202494 A1
(43) Pub. Date: Aug. 13, 2009

(54) Combined use of glp-1 agonists and gastrin for regulating blood glucose levels

(76) Inventors: Antonio Cruz, Toronto (CA); Aleksandra Pastrak, Toronto (CA); Yin Hew, Singapore (SG)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 10/587,529

(22) PCT Filed: Jan. 28, 2005
(86) PCT No.: PCT/CA05/00099

§ 371 Date: Aug. 27, 2008

(30) Foreign Application Priority Data
Jan. 30, 2004 (US) 60/540803
Jan. 30, 2004 (US) 60/540804

(51) Int. Cl. A61K 35/12; A61K 38/22

(52) U.S. Cl. 424/93.7; 514/12

(57) ABSTRACT

The invention relates generally to novel compositions and methods comprising a gastrin compound. The compositions and methods provide beneficial effects, in particular sustained beneficial effects, in particular sustained beneficial effects, in particular sustained beneficial effects, in the treatment of diabetes.

### FIELD OF THE INVENTION

The invention relates generally to compositions, conjugates, and methods comprising a GLP-1 agonist and a gastrin compound, and uses thereof.

### BACKGROUND OF THE INVENTION

Glucagon-like peptide-1 (GLP-1) is a physiological incretin hormone from the lower gastrointestinal tract GLP-1 has significant physiological activities including stimulation of glucose-dependent insulin secretion, inhibition of glucagon secretion and gastric emptying, inhibition of food intake, enhancement of glucose utilization, preservation of beta cells, inhibition of beta cell apoptosis, and induction of beta cell proliferation. [See Nauck, M. A. Acta Diabetol, 1998, 35:117-129; Holst J. J. Deabetes Metab Res Rev 2002, 18:430-441; Reimer, R. A. et al, Endocrinology 142(10): 45224528; Drucker, D. J., Molecular Endocrinology, 2003, 17(2) 161-171 and http://www.glucagon.com for reviews of GLP-1.] The above stated activities of GLP-1 make it a highly desirable therapeutic agent for the treatment of many conditions and diseases including diabetes, obesity, gastric ulcers, hypertension, chronic heart failure, fluid retentive states, metabolic syndrome, and related diseases and disorders.

The citation of any reference herein is not an admission that such reference is available as prior art to the instant invention.

### SUMMARY OF THE INVENTION

The combination of a GLP-1 agonist and a gastrin compound provides beneficial effects in the prevention and/or treatment of conditions and/or diseases for which either a GLP-1 agonist or a gastrin compound have been demonstrated to have a therapeutic effect, including but not limited to diabetes, hypertension, chronic heart failure, fluid retentive states, obesity, metabolic syndrome and related diseases and disorders. Combinations of a GLP-1 agonist and a gastrin compound may be selected to provide unexpectedly additive effects or greater than additive effects i.e. synergistic effects.

A composition, conjugate, or method comprising a GLP-1 agonist and a gastrin compound employing different mechanisms to achieve maximum therapeutic efficacy, may improve tolerance to the therapy with a reduced risk of side effects that may result from higher doses or longer term monotherapies (i.e. therapies with each compound alone). A composition, conjugate, or method of the invention will permit the use of lower doses of one or both compounds with reduced adverse toxic effects of each compound. A suboptimal dosage may provide an increased margin of safety, and may also reduce the cost of a drug necessary to achieve prophylaxis and therapy. In certain aspects of the invention, the increased convenience of a single combination dosage unit may result in enhanced compliance. Other advantages of a composition, conjugate, or combination therapy may include higher stability towards degradation and metabolism, longer duration of action, and/or longer duration of action or effectiveness at particularly low doses.

Broadly stated, the invention relates to compositions, conjugates, and methods for the prevention and/or treatment of a condition and/or disease comprising a therapeutically effective amount of a GLP-1 agonist and a gastrin compound that provide beneficial effects.

A composition, conjugate, or method of the invention may provide sustained beneficial effects following treatment or termination of treatment. Prolonged efficacy may be evidenced by increased C-peptide production, increases in pancreatic insulin production, and/or about normal blood glucose levels compared with GLP-1 or gastrin alone.

In an aspect, the invention contemplates a composition, preferably a pharmaceutical composition, comprising a GLP-1 agonist and a gastrin compound that provide beneficial effects relative to each compound alone. In another aspect the invention provides a pharmaceutical composition comprising a GLP-1 agonist and a gastrin compound that provide beneficial effects, preferably sustained beneficial effects, following treatment A pharmaceutical composition may optionally comprise a pharmaceutically acceptable carrier, excipient, or vehicle.

The invention also contemplates a pharmaceutical composition in separate containers and intended for simultaneous or sequential administration to provide beneficial effects, preferably sustained beneficial effects, comprising a GLP-1 agonist and a gastrin compound, both optionally together with pharmaceutically acceptable carriers, excipients, or vehicles.

The invention further contemplates a conjugate comprising a GLP-1 agonist interacting with or linked to a gastrin compound to provide beneficial effects, preferably sustained beneficial effects discussed herein.

The invention still further contemplates methods for preparing compositions and conjugates of the invention that result in compositions and conjugates with beneficial effects, preferably sustained beneficial effects.

In an aspect of the invention, a method is provided for preparing a stable pharmaceutical composition of a GLP-1 agonist and a gastrin compound adapted to provide beneficial effects, preferably sustained beneficial effects, following treatment, comprising preparing a composition comprising the GLP-1 agonist, a gastrin compound, and a pharmaceutically acceptable carrier, excipient, or vehicle effective to physically stabilize the GLP-1 agonist.

In another aspect of the invention, a method is provided for preparing a stable pharmaceutical composition of a GLP-1 agonist comprising mixing a GLP-1 agonist, a gastrin compound, and a pharmaceutically acceptable carrier, excipient, or vehicle effective to physically stabilize the GLP-1 agonist and adapted to provide beneficial effects, preferably sustained beneficial effects.

The invention relates to a combination treatment for preventing and/or treating a condition and/or disease discussed herein in a subject comprising administering to the subject a therapeutically effective amount of at least one GLP-1 agonist and a gastrin compound to provide beneficial effects. In an aspect the invention provides a combination treatment or intervention which provides sustained beneficial effects following treatment.

The invention further relates to the use of a GLP-1 agonist and a gastrin compound, a composition, or conjugate of the invention for preventing, and/or ameliorating disease severity, disease symptoms, and/or periodicity of recurrence of a condition and/or disease described herein. The invention still further relates to the prevention and/or treatment, in a subject, of diseases and/or conditions using a GLP-1 agonist, a gastrin and a gastrin compound, a composition, or conjugate of the invention.

In an aspect, the invention provides a method for the prevention and/or intervention of a condition and/or disease discussed herein in a subject comprising administration of at least one GLP-1 agonist and at least one gastrin compound, or a composition or conjugate of the invention. A GLP-1 agonist and a gastrin compound, composition or conjugate may be directly administered to a subject or contacted with cells (e.g. stem cells or progenitor cells) and administered to a subject.

In other aspects, the invention provides a method for the prevention and/or intervention of a condition and/or disease discussed herein in a subject comprising administration of at least one GLP-1 agonist and at least one gastrin compound to a subject in need thereof to provide beneficial effects.

In another aspect, the invention provides a method for the prevention and/or intervention of a condition and/or disease discussed herein in a subject comprising co-administering at least one GLP-1 agonist and at least one gastrin compound to a subject in need thereof.

In a particular aspect, the invention relates to inducing islet neogenesis in a subject comprising contacting islet precursor cells with a GLP-1 agonist and a gastrin compound, composition, or conjugate of the invention in a sufficient amount to increase proliferation of islet precursor cells in the subject thereby inducing islet neogenesis.

In another aspect, the invention relates to a method for treating diabetes mellitus in a patient in need thereof by administering a gastrin compound and a GLP-1 agonist or a composition comprising a gastrin compound and a GLP-1 agonist in an amount sufficient to effect differentiation of the patient's pancreatic islet precursor cells to mature insulin-secreting cells and/or to stimulate insulin synthesis in existing islet cells.

The invention provides methods for treating cells using a GLP-1 agonist and gastrin compound of the invention, or compositions, or conjugates of the invention. In particular, the invention relates to a method for expanding and differentiating stem cells or progenitor cells into insulin secreting cells, enhancing proliferation of insulin secreting cells, and/or sustaining islet cells or precursor cells. Cells may be contacted with a GLP-1 agonist and a gastrin compound in culture or in a subject.

In an aspect, a method is provided for treating a condition and/or disease comprising administering a GLP-1 agonist and a gastrin compound, a composition or conjugate of the invention with a plurality of cells to a subject in need thereof to thereby produce beneficial effects, preferably sustained beneficial effects. In an embodiment, the compounds/composition/conjugate are administered systemically.

In another aspect, the invention provides a method for treating a subject with a condition and/or disease discussed herein comprising contacting ex vivo a plurality of cells with a GLP-1 agonist and a gastrin compound, or a composition or conjugate of the invention, optionally culturing the cells, and administering the cells to the subject in need thereof.

Also provided in particular aspects of the invention are methods and compositions for treating diabetes in a patient in need thereof by implanting into a diabetic patient pancreatic islet cells that have been exposed in culture to a sufficient amount of a gastrin compound and a GLP-1 agonist, or a composition or conjugate of the invention, to increase the number of pancreatic beta cells in the islets; optionally the population of pancreatic beta cells can be grown in culture for a time sufficient to expand the population of β-cells prior to transplantation.

The invention also contemplates the use of a composition comprising a combination of at least one GLP-1 agonist and at least one gastrin compound for the preparation of one or more medicament for preventing and/or treating a condition and/or disease. The invention further contemplates use of a GLP-1 agonist in combination with a gastrin compound for the manufacture of a medicament for the treatment of a condition and/or disease. Still further the invention provides use of a GLP-1 agonist for the manufacture of a medicament for the treatment of a condition and/or disease to be used in combination with a gastrin compound.

In an aspect, the invention relates to the use of synergistically effective amounts of at least one GLP-1 agonist, and at least one gastrin compound for the preparation of a medicament for preventing or treating a condition and/or disease. In another aspect, the invention relates to the use of a GLP-1 agonist and a gastrin compound for the preparation of a medicament which has a protracted profile of action relative to GLP-1(7-37) or exendin. The invention additionally provides uses of a pharmaceutical composition and a conjugate of the invention in the preparation of medicaments for the prevention and/or treatment of conditions and/or diseases. The medicaments provide beneficial effects, preferably sustained beneficial effects following treatment.

Since the present invention relates to a method of prevention and/or treatment comprising a combination of active agents which may be administered separately or as conjugates, the invention also provides a kit comprising a GLP-1 agonist and a gastrin compound, and a pharmaceutical composition, or conjugate of the invention in kit form. These and other aspects, features, and advantages of the present invention should be apparent to those skilled in the art from the following detailed description.

### DESCRIPTION OF THE DRAWING

The invention will be better understood with reference to the drawing in which:

FIG. 1 is a graph showing the effect of islet neogenesis therapy using a GLP-1 agonist and a gastrin compound (INT-2) on fasting blood glucose in acutely diabetic NOD mice. The Figure illustrates that the combination of GLP-1 (300 μg/kg) and Gastrin (3 μg/kg) treatment reversed hyperglycemia and prevented death in NOD mice.

FIG. 2 are graphs showing individual fasting blood glucose levels in acutely diabetic NOD mice treated with the combination of GLP-1 (300 μg/kg) and Gastrin (3 μg/kg).

FIG. 3 is a graph showing that a GLP-1 and Gastrin combination treatment restores pancreatic insulin levels in a NOD mouse model.

FIG. 4 is a graph showing the correlation between pancreatic insulin content and fasting blood glucose levels.

FIG. 5 shows insulin stained cells in acutely diabetic NOD mice treated with vehicle and GLP-1 and Gastrin.

FIG. 6 shows staining of islet cells from the pancreatic duct in NOD mice treated with vehicle and GLP-1 and Gastrin.

### GLOSSARY

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. Thus, for example, reference to a composition containing “a compound” includes a mixture of two or more compounds. The term “about” means plus or minus 0.1 to 50%, 5-50%, or 10-40%, preferably 10-20%, more preferably 10% or 15%, of the number to which reference is being made.

Selected compounds described herein contain one or more asymmetric centers and may give rise to enantiomers, diasteriomers, and other stereoisomeric forms which may be defined in terms of absolute stereochemistry as (R)- or (S)-. Therefore, the invention includes all such possible diasteriomers and enantiomers as well as their racemic and optically pure forms. Optically active (R)- and (S)-isomers may be prepared using chiral synthons or chiral reagents; or resolved using conventional techniques. When the compounds described herein contain centers of geometric asymmetry, and unless specified otherwise, it is intended that the compounds include both B and A geometric isomers. All tautomeric forms are intended to be included within the scope of the invention.

The terms “subject”, “individual” or “patient” refer to an animal including a warm-blooded animal such as a mammal, which is afflicted with or suspected of having or being pre-disposed to a condition and/or disease as described herein. Preferably, the terms refer to a human. The terms also include domestic animals bred for food, sport, or as pets, including horses, cows, sheep, poultry, fish, pigs, cats, dogs, and zoo animals. The methods herein for use on subjects individuals/patients contemplate prophylactic as well as curative use. Typical subjects for treatment include persons susceptible to, suffering from or that have suffered a condition and/or disease discussed herein.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. The use of such media and agents for an active substance is well known in the art. In certain aspects of the invention, a carrier, excipient, or vehicle is selected to stabilize a GLP-1 agonist.

“Pharmaceutically acceptable salt(s),” includes salts of acidic or basic groups which may be present in the compounds suitable for use in the present invention. Examples of pharmaceutically acceptable salts include sodium, calcium and potassium salts of carboxylic acid groups and hydrochloride salts of amino groups. Other pharmaceutically acceptable salts of amino groups are hydrobromide, sulfate, hydrogen sulfate, phosphate, hydrogen phosphate, dihydrogen phosphate, acetate, succinate, citrate, tartrate, lactate, mandelate, methanesulfonate (mesylate) and p-toluenesulfonate (tosylate) salts.

The terms “preventing and/or treating”, “prevention and/or treatment”, or “prevention and/or intervention” refer to the administration to a subject of biologically active agents either before or after onset of a condition and/or disease. A treatment may be either performed in an acute or chronic way. In particular, prevention includes the management and care of a subject at risk of developing a condition and/or disease discussed herein prior to the clinical onset of the condition and/or disease. Treatment or intervention refers to the management and care of a subject at diagnosis or later. An objective of prevention, treatment, or intervention is to combat the condition and/or disease and includes administration of the active compounds to prevent or delay the onset of the symptoms or complications, or alleviating the symptoms or complications, or eliminating or partially eliminating the condition and/or disease.

A “beneficial effect” refers to an effect of a combination of a GLP-1 agonist and a gastrin compound, or composition or conjugate thereof, that is greater than the effect of either of the compounds alone. The beneficial effect includes favorable pharmacological and/or therapeutic effects, and improved pharmacokinetic properties and biological activity. A beneficial effect may be an additive effect or synergistic effect. In preferred embodiments of the invention, beneficial effects include but are not limited to the following: reduced or absent islet inflammation, decreased disease progression, increased survival, or elimination or partial elimination of a condition and/or disease. In a particularly preferred embodiment, the beneficial effect is a “sustained beneficial effect” where the beneficial effect is sustained for a prolonged period of time after termination of treatment. In an embodiment, one or more of the aforementioned effects are sustained for a prolonged period of time after termination of treatment. A beneficial effect may be sustained for at least about 2, 4, 6, 8, 10, 2 to 4 weeks, 2 to 6 weeks, 2 to 8 weeks, 2 to 12 weeks, 2 to 24 weeks, 2 weeks to 12 months, and 2 weeks to 18 months following treatment. The period of time a beneficial effect is sustained may correlate with the duration and timing of the treatment A subject may be treated continuously for about 2 to 8 weeks, 2 to 12 weeks, 2 to 16 weeks, 2 weeks to 6 months, 2 weeks to 12 months, or periodically. A sustained beneficial effect may manifest as one or more of increased C-peptide production, increased pancreatic insulin production, and/or, about normal or low blood glucose levels for a prolonged period following treatment.

The beneficial effect may be a statistically significant effect in terms of statistical analysis of an effect of the two compounds versus the effects of each of the compounds. “Statistically significant” or “significantly different” effects or levels with two compounds compared with each compound alone may represent levels that are higher or lower than a standard. In embodiments of the invention, the difference may be 1.5, 2, 3, 4, 5, or 6 times higher or lower compared with the effect obtained with each compound alone.

An “additive effect” of a GLP-1 agonist and a gastrin compound refers to an effect that is equal to the sum of the effects of the two individual compounds.

A “synergistic effect” of a GLP-1 agonist and a gastrin compound refers to an effect that is greater than the additive effect which results from the sum of the effects of the two individual compounds.

“Combination treatment”, “combination therapy”, and “administering in combination” are used interchangeably herein and mean that the active ingredients are administered concurrently to a patient being treated. When administered in combination each component may be administered at the same time, or sequentially in any order at different points in time. Therefore, each component may be administered separately, but sufficiently close in time to provide the desired effect, in particular a beneficial, additive, or synergistic effect. The first compound may be administered in a regimen which additionally comprises treatment with the second compound. In certain embodiments, the term refers to administration of a GLP-1 agonist and a gastrin compound to a patient within one year, including separate administration of two medicaments each containing one of the compounds as well as simultaneous administration whether or not the two compounds are combined in one formulation or whether they are two separate formulations.

A “medicament” refers to a pharmaceutical composition suitable for administration of a pharmaceutically active compound(s) (e.g. a GLP-1 agonist and/or a gastrin compound) to a patient.

“Therapeutically effective amount” relates to the amount or dose of active compounds (e.g. GLP-1 agonist and gastrin compound), compositions or conjugates of the invention that will lead to one or more desired beneficial effects, preferably one or more sustained beneficial effects. A “therapeutically effective amount” can provide a dosage which is sufficient in order for prevention and/or treatment of a subject to be effective compared with no treatment.

“Synergistically effective amount” relates to the amount of dose of active compounds (e.g. GLP-1 agonist and gastrin compound), compositions or conjugates of the invention that will provide a synergistic effect, in particular a synergistic beneficial effect.

“Suboptimal dose” or suboptimal dosage” refers to a dose or dosage of an active compound which is less than the optimal dose or dosage for that compound when used in monotherapy.

The terms “associated”, “linked”, “interact”, “interaction”, or “interacting” refer to any physical association between molecules. The terms preferably refers to a stable association between two molecules due to, for example, electrostatic, hydrophobic, ionic, hydrogen-bond interactions, or covalent interactions. Certain interacting or associated molecules interact only after one or more of them has been activated.

In the present context, “a GLP-1 agonist” is understood to refer to any compound, including peptides and non-peptide compounds, which fully or partially activate the human GLP-1 receptor. In a preferred embodiment, the “GLP-1 agonist” is any peptide or non-peptide small molecule that binds to a GLP-1 receptor, preferably with an affinity constant (K_{D}) or a potency (EC₅₀) of below 1 μM e.g. below 100 nM as measured by methods known in the art (see e.g. WO 98/08871) and exhibits insulinotropic activity, where insulinotropic activity may be measured in vivo or in vitro assays known to those of ordinary skill in the art. For example, the GLP-1 agonist may be administered to an animal and the insulin concentration measured over time.

In one embodiment, the GLP-1 agonist is selected from the group consisting of GLP-1(7-36)-amide, GLP-1(7-37), a GLP-1(7-36)-amide analogue, a GLP-1(7-37) analogue, or a derivative of any of these.

In the present application, the designation “an analogue” is used to designate a peptide wherein one or more amino acid residues of the parent peptide have been substituted by another amino acid residue and/or wherein one or more amino acid residues of the parent peptide have been deleted and/or wherein one or more amino acid residues have been added to the parent peptide. Such addition can take place either at the N-terminal end or at the C-terminal end of the parent peptide or both. Typically “an analogue” is a peptide wherein 6 or less amino acids have been substituted and/or added and/or deleted from the parent peptide, more preferably a peptide wherein 3 or less amino acids have been substituted and/or added and/or deleted from the parent peptide, and most preferably, a peptide wherein one amino acid has been substituted and/or added and/or deleted from the parent peptide.

In the present application, “a derivative” is used to designate a peptide or analogue thereof which is chemically modified by introducing e.g. ester, alkyl or lipophilic functionalities on one or more amino acid residues of the peptide or analogue thereof.

Methods for identifying GLP-1 agonists are described in WO 93/19175 (Novo Nordisk A/S) and examples of suitable GLP-1 analogues and derivatives which can be used according to the present invention includes those referred to in WO 99/43705 (Novo Nordisk A/S), WO 99/43706 (Novo Nordisk A/S), WO 99/43707 (Novo Nordisk A/S), WO 98/08871 (Novo Nordisk A/S), WO 99/43708 (Novo Nordisk A/S), WO 99/43341 (Novo Nordisk A/S), WO 87/06941 (The General Hospital Corporation), WO 90/11296 (The General Hospital Corporation), WO 91/11457 (Buckley et al.), WO 98/43658 (Eli Lilly & Co.), EP 0708179-A2 (Eli Lilly & Co.), BP 0699686-A2 (Eli Lilly & Co.), WO 01/98331 (Eli Lilly & Co).

In one embodiment, the GLP-1 agonist is a derivative of GLP-1(7-36)-amide, GLP-1(7-37), a GLP-1(7-36)-amide analogue or a GLP-1(7-37) analogue, which comprises a lipophilic substituent.

In this embodiment of the invention, the GLP-1 derivative preferably has three lipophilic substituents, more preferably two lipophilic substituents, and most preferably one lipophilic substituent attached to the parent peptide (ie GLP-1(7-36)-amide, GLP-1(7-37), a GLP-1(7-36)-amide analogue or a GLP-1(7-37) analogue), where each lipophilic substituent(s) preferably has 4-40 carbon atoms, more preferably 8-30 carbon atoms, even more preferably 8-25 carbon atoms, even more preferably 12-25 carbon atoms, and most preferably 14-18 carbon atoms.

In one embodiment, the lipophilic substituent comprises a partially or completely hydrogenated cyclopentanophenathrene skeleton.

In another embodiment, the lipophilic substituent is a straight-chain or branched alkyl group.

In yet another embodiment, the lipophilic substituent is an acyl group of a straight-chain or branched fatty acid. Preferably, the lipophilic substituent is an acyl group having the formula CH₃(CH)ₙCO—, wherein n is an integer from 4 to 38, preferably an integer from 12 to 38, and most preferably is CH₃(CH₂)₁₂CO, CH₃(CH₂)₁₄CO—, CH₃(CH₂)₁₆CO—, CH₃(CH₂)₁₈CO—, CH₃(CH)₂₀CO— and CH₃(CH)₂₂CO—. In a more preferred embodiment, the lipophilic substituent is tetradecanoyl. In a most preferred embodiment, the lipophilic substituent is hexadecanoyl.

In a further embodiment of the present invention, the lipophilic substituent has a group which is negatively charged such as a carboxylic acid group. For example, the lipophilic substituent may be an acyl group of a straight-chain or branched alkane a,w-dicarboxylic acid of the formula HOOC(CH₂)ₘCO—, wherein m is an integer from 4 to 38, preferably an integer from 12 to 38, and most preferably is HOOC(CH₂)₁₄CO—, HOOC(CH₂)₁₆CO—, HOOC(CH₂)₁₈CO—, HOOC(CH₂)₂₀CO— or HOOC(CH₂)₂₂CO—.

In the GLP-1 derivatives of the invention, the lipophilic substituent(s) contain a functional group which can be attached to one of the following functional groups of an amino acid of the parent GLP-1 peptide:

(a) the amino group attached to the alpha-carbon of the N-terminal amino acid,
(b) the carboxy group attached to the alpha-carbon of the C-terminal amino acid,
(c) the epsilon-amino group of any Lys residue,
(d) the carboxy group of the R group of any Asp and Glu residue,
(e) the hydroxy group of the R group of any Tyr, Ser and Thr residue,
(f) the amino group of the R group of any Trp, Asn, Gln, Arg, and His residue, or
(g) the thiol group of the R group of any Cys residue.

In one embodiment, a lipophilic substituent is attached to the carboxy group of the R group of any Asp and Glu residue.

In another embodiment, a lipophilic substituent is attached to the carboxy group attached to the alpha-carbon of the C-terminal amino acid.

In a most preferred embodiment, a lipophilic substituent is attached to the epsilon-amino group of any Lys residue.

In a preferred embodiment of the invention, the lipophilic substituent is attached to the parent GLP-1 peptide by means of a spacer. A spacer must contain at least two functional groups, one to attach to a functional group of the lipophilic substituent and the other to a functional group of the parent GLP-1 peptide.

In one embodiment, the spacer is an amino acid residue except Cys or Met, or a dipeptide such as Gly-Lys. For purposes of the present invention, the phrase “a dipeptide such as Gly-Lys” means any combination of two amino acids except Cys or Met, preferably a dipeptide wherein the C-terminal amino acid residue is Lys, His or Trp, preferably Lys, and the N-terminal amino acid residue is Ala, Arg, Asp, Asn, Gly, Glu, Gln, Ile, Leu, Val, Phe, Pro, Ser, Tyr, Thr, Lys, His and Trp. Preferably, an amino group of the parent peptide forms an amide bond with a carboxylic group of the amino acid residue or dipeptide spacer, and an amino group of the amino acid residue or dipeptide spacer forms an amide bond with a carboxyl group of the lipophilic substituent.

Preferred spacers are lysyl, glutamyl, asparagyl, glycyl, beta-alanyl and gamma-aminobutanoyl, each of which constitutes an individual embodiment. Most preferred spacers are glutamyl and beta-alanyl. When the spacer is Lys, Glu or Asp, the carboxyl group thereof may form an amide bond with an amino group of the amino acid residue, and the amino group thereof may form an amide bond with a carboxyl group of the lipophilic substituent. When Lys is used as the spacer, a further spacer may in some instances be inserted between the e-amino group of Lys and the lipophilic substituent. In one embodiment, such a further spacer is succinic acid which forms an amide bond with the c-amino group of Lys and with an amino group present in the lipophilic substituent. In another embodiment such a further spacer is Glu or Asp which forms an amide bond with the e-amino group of Lys and another amide bond with a carboxyl group present in the lipophilic substituent, that is, the lipophilic substituent is a N^{e}-acylated lysine residue.

In another embodiment, the spacer is an unbranched alkane a,w-dicarboxylic acid group having from 1 to 7 methylene groups, which spacer forms abridge between an amino group of the parent peptide and an amino group of the lipophilic substituent. Preferably, the spacer is succinic acid.

In a further embodiment, the lipophilic substituent with the attached spacer is a group of the formula CH₃(CH₂)ₚNH—CO(CH₂)_{q}CO—, wherein p is an integer from 8 to 33, preferably from 12 to 28 and q is an integer from 1 to 6, preferably 2.

In a further embodiment, the lipophilic substituent with the attached spacer is a group of the formula CH₃(CH₂)ᵣCO—NHCH(COOH)(CH₂)₂CO—, wherein r is an integer from 4 to 24, preferably from 10 to 24.

In a further embodiment, the lipophilic substituent with the attached spacer is a group of the formula CH₃(CH₂)ₛCO—NHCH((CH₂)₂COOH)CO—, wherein s is an integer from 4 to 24, preferably from 10 to 24.

In a further embodiment, the lipophilic substituent is a group of the formula COOH(CH₂)ₜCO— wherein t is an integer from 6 to 24.

In a further embodiment, the lipophilic substituent with the attached spacer is a group of the formula —NHCH(COOH)(CH₂)₄NH—CO(CH₂)ᵤCH₃, wherein u is an integer from 8 to 18.

In a further embodiment, the lipophilic substituent with the attached spacer is a group of the formula CH₃(CH₂)ᵥCO—NH—(CH₂)_{z}—CO, wherein v is an integer from 4 to 24 and z is an integer from 1 to 6.

In a further embodiment, the lipophilic substituent with the attached spacer is a group of the formula —NHCH(COOH)(CH₂)₄NH—COCH((CH₂)₂COOH)NH—CO(CH₂)_{w}CH₃, wherein w is an integer from 10 to 16.

In a further embodiment, the lipophilic substituent with the attached spacer is a group of the formula —NHCH(COOH)(CH₂)₄NH—CO(CH₂)₂CH(COOH)NHCO(CH₂)ₓCH₃, wherein x is zero or an integer from 1 to 22, preferably 10 to 16.

In yet another embodiment the GLP-1 agonist is Arg³⁴, Lys²⁶(N^{ε}-(γ-Glu(N^{α}-hexadecanoyl)))-GLP-1(7-37).

In yet another embodiment the GLP-1 agonist is selected from the group consisting of Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²-GLP-1(7-37), analogues thereof and derivatives of any of these.

In yet another embodiment the GLP-1 agonist is selected from the group consisting of Arg²⁶-GLP-1(7-37); Arg³⁴-GLP-1(7-37); Lys³⁶-GLP-1(7-37); Arg^{26,34}Lys³⁶-GLP-1(7-37); Arg^{26,34}-GLP-1(7-37); Arg^{26,34}Lys⁴⁰-GLP-1(7-37); Arg²⁶Lys³⁶-GLP-1(7-37); Arg³⁴Lys³⁶-GLP-1(7-37); Val⁸Arg²²-GLP-1(7-37); Met⁸Arg²²-GLP-1(7-37); Gly⁸His²²-GLP-1(7-37); Val⁸His²²-GLP-1(7-37); Met⁸His²²-GLP-1(7-37); His³⁷′-GLP-1(7-37); Gly⁸-GLP-1(7-37); Val⁸-GLP-1(7-37); Met⁸-GLP-1(7-37); Gly⁸Asp²²-GLP-1(7-37); Val⁸Asp²²-GLP-1(7-37); Met⁸Asp²²-GLP-1(7-37); Gly⁸Glu²²-GLP-1(7-37); Val⁸Glu²²-GLP-1(7-37); Met⁸Glu²²-GLP-1(7-37); Gly⁸Lys²²-GLP-1(7-37); Val⁸Lys²²-GLP-1(7-37); Met⁸Lys²²-GLP-1(7-37); Gly⁸Arg²²-GLP-1(7-37);

Val⁸Lys²²His³⁷-GLP-1(7-37); Gly⁸Glu²²His³⁷-GLP-1(7-37); Val⁸Glu²²His³⁷-GLP-1(7-37); Met⁸Glu²²His³⁷-GLP-1(7-37); Gly⁸Lys²²His³⁷-GLP-1(7-37); Met⁸Lys²²His³⁷-GLP-1(7-37); Gly⁸Arg²²His³⁷-GLP-1(7-37); Val⁸Arg²²His³⁷-GLP-1(7-37); Met⁸Arg²²His³⁷-GLP-1(7-37); Gly⁸His²²His³⁷-GLP-1(7-37); Val⁸His²²His³⁷-GLP-1(7-37); Met⁸His²²His³⁷-GLP-1(7-37); Gly⁸His³⁷-GLP-1(7-37); Val⁸His³⁷-GLP-1(7-37); Met⁸His³⁷-GLP-1(7-37); Gly⁸Asp²²His³⁷-GLP-1(7-37); Val⁸Asp²²His³⁷-GLP-1(7-37); Met⁸Asp²²His³⁷-GLP-1(7-37); Arg²⁶-GLP-1(7-36)-amide; Arg³⁴-GLP-1(7-36)-amide; Lys³⁶-GLP-1(7-36)-amide; Arg^{26,34}Lys³⁶GLP-1(7-36)-amide; Arg³⁴-GLP-1(7-36)-amide; Arg^{26,34}Lys⁴⁰-GLP-1(7-36)-amide; Arg²⁶Lys³⁶-GLP-1(7-36)-amide; Arg³⁴Lys³⁶-GLP-1(7-36)-amide; Gly⁸-GLP-1(7-36)-amide; Val⁸-GLP-1(7-36)-amide; Met⁸-GLP-1(7-36)-amide; Gly⁸Asp²²-GLP-1(7-36)-amide; Gly⁸Glu²²His³⁷-GLP-1(7-36)-amide; Val⁸Asp²²-GLP-1(7-36)-amide; Met⁸Asp²²-GLP-1(7-36)-amide; Gly⁸Glu²²-GLP-1(7-36)-amide; Val⁸Glu²²-GLP-1(7-36)-amide; Met⁸Glu²²-GLP-1(7-36)-amide; Gly⁸Lys²²-GLP-1(7-36)-amide; Val⁸Lys²²-GLP-1(7-36)-amide; Met⁸Lys²²-GLP-1(7-36)-amide; Gly⁸His²²His³⁷-GLP-1(7-36)-amide; Gly⁸Arg²²-GLP-1(7-36)-amide; Val⁸Arg²²-GLP-1(7-36)-amide; Met⁸Arg²²-GLP-1(7-36 amide; Gly⁸His²²-GLP-1(7-36)-amide; Val⁸His²²-GLP-1(7-36)-amide; Met⁸His²²-GLP-1(7-36)-amide; His³⁷-GLP-1(7-36)-amide; Val⁸Arg²²His³⁷-GLP-1(7-36)-amide; Met⁸Arg²²His³⁷-GLP-1(7-36)-amide; Gly⁸His³⁷-GLP-1 (7-36)-amide; Val⁸His³⁷-GLP-1(7-36)-amide; Met⁸His³⁷-GLP-1(7-36)-amide; Gly⁸Asp²²His³⁷-GLP-1(7-36)-amide; Val⁸Asp²²His³⁷-GLP-1(7-36)-amide; Met⁸Asp²²His³⁷-GLP-1(7-36)-amide; Val⁸Glu²²His³⁷-GLP-1(7-36)-amide; Met⁸Glu²²His³⁷-GLP-1(7-36)-amide; Gly⁸Lys²² His³⁷-GLP-1(7-36)-amide; Val⁸Lys²²His³⁷-GLP-1(7-36)-amide; Met⁸Lys²²His³⁷-GLP-1(7-36)-amide; Gly⁸Asp²²His³⁷-GLP-1(7-36)-amide; Val⁸His²²His³⁷-GLP-1(7-363)-amide; Met⁸His²²His³⁷-GLP-1(7-36)-amide; and derivatives thereof.

In yet another embodiment the GLP-1 agonist is selected from the group consisting of Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷-GLP-1(7-37), Val⁸Glu²²Ile³³-GLP-1(7-37), Val⁸Trp¹⁶Glu²²Val²⁵Ile³³-GLP-1(7-37), Val⁸Trp¹⁶Glu²²Ile³³-GLP-1(7-37), Val⁸Glu²²Val²⁵Ile³³-GLP-1(7-37), Val⁸Trp¹⁶Glu²²Val²⁵-GLP-1(7-37), analogues thereof and derivatives of any of these.

In yet another embodiment the GLP-1 agonist is a stable GLP-1 analogue/derivative. Throughout this application a “stable GLP-1 analogue/derivative” means a GLP-1 analogue or a derivative of a GLP-1 analogue which exhibits an in vivo plasma elimination half-life of at least 10 hours in man, as determined by the method described below. Examples of stable GLP-1 analogue/derivatives can be found in WO 98/08871 and WO 99/43706. The method for determination of plasma elimination half-life of a compound in man is: The compound is dissolved in an isotonic buffer, pH 7.4, PBS or any other suitable buffer. The dose is injected peripherally, preferably in the abdominal or upper thigh. Blood samples for determination of active compound are taken at frequent intervals, and for a sufficient duration to cover the terminal elimination part (e.g. Pre-dose, 1, 2, 3, 4, 5, 6, 7, 8, 10, 12, 24 (day 2), 36 (day 2), 48 (day 3), 60 (day 3), 72 (day 4) and 84 (day 4) hours post dose). Determination of the concentration of active compound is performed as described in Wilken et al., Diabetologia 43(51):A143, 2000. Derived pharmacokinetic parameteres are calculated from the concentration-time data for each individual subject by use of non-compartmental methods, using the commercially available software WinNonlin Version 2.1 (Pharsight, Cary, N.C., USA). The terminal elimination rate constant is estimated by log-linear regression on the terminal log-linear part of the concentration-time curve, and used for calculating the elimination half-life.

Stable GLP-1 analogues and derivatives are disclosed in WO 98/08871 (analogues with lipophilic substituent) and in WO 02/46227 (analogues fused to serum albumin or to Fc portion of an Ig).

In another embodiment, The GLP-1 agonist is formulated so as to have a half-life in man, as discussed above, of at least 10 hours. This may be obtained by sustained release formulations known in the art.

In yet another embodiment the GLP-1 agonist is exendin-4 or exendin-3, an exendin-4 or exendin-3 analogue or a derivative of any of these.

Examples of exendins as well as analogues, derivatives, and fragments thereof to be included within the present invention are those disclosed in WO 97/46584, U.S. Pat. No. 5,424,286 and WO 01/04156. U.S. Pat. No. 5,424,286 describes a method for stimulating insulin release with an exendin polypeptide. The exendin polypeptides disclosed include HGBGTFTSDLSKQMEBEAVRLFIEWLKNGGX [SEQ ID NO. 20]; wherein X=P or Y, and HX1X2GTFITSDLSKQMBBEAVRLFIEWLKNGGPSSGAPPPS [SEQ ID NO. 21]; wherein X1X2=SD (exendin-3) or GE (exendin-4)). WO 97/46584 describes truncated versions of exendin peptide(s). The disclosed peptides increase secretion and biosynthesis of insulin, but reduce those of glucagon. WO 01/04156 describes exendin-4 analogues and derivatives as well as the preparation of these molecules. Exendin-4 analogues stabilized by fusion to serum albumin or Fc portion of an Ig are disclosed in WO 02/46227.

In one embodiment, the exendin-4 analogue is HGEGTFTSDLSKQMEEEAVRLFIBWLKNGGPSSOAPPSKKKKKK [SEQ ID NO. 22].

In yet another embodiment the GLP-1 agonist is a stable exendin-4 analogue/derivative. The term “stable exendin-4 analogue/derivative”, as used herein refers to an exendin-4(1-39) analogue or a derivative of an exendin-4(1-39) analogue which exhibits an in vivo plasma elimination half-life of at least 10 hours in man, as determined by the method described above for a “stable GLP-1 analogue/derivative”.

In still another embodiment, the GLP-1 agonist is Aib^{8,35} GLP-1(7-36) amide (Aib=a-amino isobutyric acid).

In still another embodiment, the GLP-1 agonist is Ser³⁸Lys^{39,40,41,42,43,44}-Exendin-4(1-39)-amide.

In still another embodiment the GLP-1 agonist is selected from the non-peptide small molecule GLP-1 agonists disclosed in WO 00/42026.

An amino acid portion of a GLP-1 agonist can be prepared by a variety of methods known in the art such as solid-phase synthesis, purification of GLP-1 agonists from natural sources, recombinant technology, or a combination of these methods. See for example, U.S. Pat. Nos. 5,188,666, 5,120,712, 5,523,549, 5,512,549, 5,977,071, 6,191,102, Dugas and Penney 1981, Merrifield, 1962, Stewart and Young 1969, and the references cited herein. GLP-1 agonist derivatives can be produced by appropriate derivatization of an appropriate backbone produced, for example, by recombinant DNA technology or peptide synthesis (e.g. Merrifield-type solid phase synthesis) using methods known in the art of peptide synthesis and peptide chemistry.

A “gastrin compound” is understood to refer to any compound, including peptides and non-peptide compounds, which fully or partially associate with and/or activate the gastrin/CCK_{B} receptor. In aspects of the invention, a gastrin compound is selected that has a suitable IC₅₀, for example an IC₅₀ of about ˜0.7 nM at a gastrin/CCK_{B} receptor, as measured by methods known in the art (see Singh et al (1995) J. Biol. Chem. 270: 8429-8438, and Kopin et al (1995) J. Biol. Chem. 270: 5019-5023 describing in vitro cell growth assays, and receptor binding assays as described in Singh et al (1995) J. Biol. Chem. 270:8429-8438, and Kopin et al (1995) J. Biol. Chem. 270: 5019-5023). A gastrin compound may also be selected based on other criteria such as activity, half-life etc. as discussed herein.

A “gastrin compound” includes, without limitation, the various forms of gastrin, such as gastrin 71, gastrin 52, gastrin 34 (big gastrin), gastrin 17 (little gastrin), gastrin 14, and gastrin 8 (mini gastrin), pentagastrin, tetragastrin, and fragments, analogs, and derivatives thereof. Sequences for gastrins including big gastrin-34 (Bonato et al, 1986, Life Science 39:959) and small gastrin-17 (Bentley et al (1966) Nature 209:583) are known in the art, and some are shown in SEQ ID NOs. 11 to 18. In particular, sequences for gastrins include gastrin 71 of SEQ ID NO. 15, gastrin 52 of SEQ ID NO. 16, gastrin 34 (big gastrin) of SEQ ID NO. 11 or 12, gastrin 17 (little gastrin) of SEQ ID NO. 13 or 14, gastrin 14 of SEQ ID NO. 17, and gastrin 6 of SEQ ID NO.18 or 19. Gastrin-34 is essentially an extension of an amino acid sequence at the N-terminal end of gastrin-17. Big gastrin is cleaved in vivo to release gastrin-17. Glp at the N-terminal end of a gastrin is pyroglutamate, which is a naturally cyclized form of glutamate. In various embodiments, where cysteine or lysine is added to a terminus of gastrin having a pyroglutamate, the pyroglutamate is replaced with a glutamate, or the pyroglutamate is deleted. A gastrin 34 or gastrin-17 may be used in the invention where there is a methionine or a leucine at position 15, as shown in SEQ ID NOs: 6-9 herein.

Examples of gastrin compounds that may be used in the present invention include the compounds disclosed in U.S. Pat. No. 6,288,301. In some applications of the invention, a gastrin compound may be selected that is a peptide or non-peptide agonist or partial agonist of the gastrin receptor such as A71378 (Lin et al., Am. J. Physiol. 258 (4 Pt 1): G648, 1990). In some applications of the invention, a gastrin compound may be selected that is a gastrin/CCK_{B} receptor ligand including but not limited to cholecystokinin (CCK) such as CCK 58, CCK 33, CCK 22, CCK 12 and CCK 8; and the like.

In certain aspects, a gastrin compound may be an active analog, fragment or other modification which, for example, share amino acid sequence with an endogenous mammalian gastrin, for example, share 60% sequence identity, or 70% identity, or 80% identity. Such compounds also include substances that increase the secretion of endogenous gastrins, cholecystokinins or similarly active peptides from sites of tissue storage. Examples of these are the gastric releasing peptide, omeprazole which inhibits gastric acid secretion, and soya bean trypsin inhibitor which increases CCK stimulation.

A “gastrin compound” includes a modified form of a gastrin, including but not limited to a modified form of gastrin 71 [SEQ ID NO. 15], gastrin 52 [SEQ ID NO. 16], gastrin 34 (big gastrin) [SEQ ID NO. 11 or 12], gastrin 17 (little gastrin) [SEQ ID NO. 13 or 14], gastrin 14 [SEQ ID NO. 17], gastrin 8, gastrin 6 [SEQ ID NO:18], pentagastrin, and tetragastrin. A modified gastrin preferably comprises TrpMetAspPhe-NH₂ [SEQ ID NO. 26] or TrpLeuAspPhe-NH₂ [SEQ ID NO.27].

In aspects of the invention a modified gastrin comprises at least amino acids 1-34, 18-34 or 29-34 of SEQ ID NO. 11 or 12, or amino acids 1-17, 2-17, 12-17, or 14-17 of SEQ ID NO. 13 or 14.

Modified gastrin compounds for use in the present invention comprise the modified gastrin compounds described in PCT/CA03/01778, U.S. Ser. No. 10/719,450 and U.S. Application Ser. No. 60/519,933 incorporated in their entirety by reference.

In particular, a modified gastrin can be a gastrin derivative or analogue comprising a minimal sequence of 6 amino acids (from the C-terminal end) of a gastrin, in particular amino acid residues 1 to 34, 18 to 34 or 29-34 of SEQ ID NO: 11 or 12, or amino acid residues 1-17, 2-17, 12-17, or 14-17 of SEQ ID NO. 13 or 14, and comprising a reactive group capable of undergoing an addition reaction. Examples of reactive groups include without limitation thiols, alpha amino groups, epsilon amino groups, carboxyl groups or aromatic rings. A reactive group is generally capable of linking a gastrin sequence, directly or indirectly via a crosslinking agent and/or spacer region, to a carrier.

A reactive group may be introduced by adding or substituting an amino acid comprising a reactive group, for example by adding a cysteine or lysine. Therefore, a modified gastrin may comprise a gastrin sequence (e.g. gastrin-34 or gastrin 17) wherein at least one reactive amino acid (e.g. cysteine or lysine) is added or substituted. The addition of a reactive amino acid can be at a terminal region, in particular an N-terminal region.

A modified gastrin may also optionally comprise a spacer. A spacer can interact with a reactive group, for example, an amino acid comprising a reactive group. A spacer can be one or more amino acids, peptides, peptidomimetics, or small organic molecules. A spacer can comprise at least one amino acid, preferably at least two, three, four or five amino acids and in certain embodiments it is a sequence of several amino acids, including without limitation alanine or glycine. A spacer can comprise alternating amino acids (e.g. glycine and/or alanine), non-alternating amino acids, a random sequence or a particular sequence. By way of example, a spacer can be synthesized as part of, or may be chemically attached to an amino acid of a gastrin sequence.

A modified gastrin may optionally comprise a cross-linking agent. A cross-linking agent may comprise a homobifunctional or heterobifunctional portion for interaction directly or indirectly with a gastrin, spacer and/or a reactive group. A cross-linking agent may interact with a gastrin sequence or a spacer, or it may be added to a reactive group at the end (in particular N-terminus) of a modified gastrin.

A cross-linking agent can be any agent that can link a gastrin sequence and a carrier directly or via a spacer. Examples of homobifunctional crosslinking agents include without limitation amino group directed homobifunctional cross-linking reagents such as bisimidates (e.g. methyl acetimidate-HCl), bifunctional aryl halides (e.g. 1,5-dichloro-2,4-dinitrobenzene), bifunctional acylating agents (e.g. diisocyanates), bifunctional sulfonyl halides (e.g. phenol-2,4-disulfonyl-chloride), bifunctional acylazides (e.g. tartryl diazide), dialdehydes (e.g. glutaraldehyde), and diketones (e.g. 2,5-hexanedione). Examples of heterobifunctional crosslinkers include amino and sulfhydryl group directed bifunctional reagents (e.g. N-succinimidyl-3-(2-pyridyldithio propionate, carboxyl and either sulfhydryl or amino group directed bifunctional reagents (e.g. p-nitrophenyl diazoacetate), and carbonyl and sulfhydryl group directed bifunctional reagents (e.g. 1-(aminooxy)-4 [3-nitro-2-pyridyl)dithio)]butane).

A modified gastrin can optionally comprise a carrier which may be a polymer. A carrier may be a polymer of amino acids (proteins), sugars (polysaccharides), nucleosides, synthetic polymers and mixtures thereof. A protein carrier may be a protein found in the circulatory system. Examples of protein carriers found in the circulatory system, in particular the human circulatory system, include without limitation plasma components such as serum, purified serum proteins such as albumin (in particular human serum albumin), transferrin, or an immunoglobulin, red blood cell proteins such as glycophorin A and AE-1, sugar binding proteins such as a lectin, inactivated enzymes, phosphate and sulphate binding proteins, and lipid binding proteins. Examples of other suitable polymeric carriers include without limitation cellulose and derivatives thereof, starch and derivatives thereof, heparin and derivatives thereof, and synthetic polymers such as polyethylene glycol (PEG) and dextran, and derivatives thereof. Carriers may be attached to a gastrin or spacer by way of reactive groups on, or introduced to, the carrier, gastrin, and/or spacer. For example, carriers can be covalently attached to reactive groups (such as thiol groups, alpha and epsilon amino groups, carboxyl groups or aromatic groups) on a gastrin or spacer which may be present or added by chemical modification of the gastrin or spacer.

In certain aspects of the invention, a modified gastrin can comprise a gastrin of SEQ ID NOS 11, 12, 13, 14, 17, or 18 and a carrier.

A group of modified gastrin compounds include compounds having an amino acid sequence comprising from the amino terminus Z-Yₘ-Xₙ-AA₁-AA₂-AA₃-AA₄-AA₅-AA₆, wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr and wherein AA₆ is optionally amidated; Z is a carrier, in particular a polymer and when the polymer is a protein Z is an amino acid sequence; Yₘ is an optional spacer region comprising m amino acid residues of a small neutral amino acid including but not limited to serine and alanine, and X is any consecutive portion of residues 1-28 (=n) of SEQ ID NO: 11 or 12 or 1-11 of SEQ ID. NO. 13 or 14, providing that the gastrin compound binds a gastrin/CCK_{B} receptor. Generally, m is 0 to about 20 residues. In an aspect Z is a protein, in particular a protein of the circulatory system, more particularly a serum protein, still more particularly albumin, most particularly human serum albumin.

In embodiments, X is one or more amino acid residues from position 18 to position 28 of SEQ ID NO.: 11. Therefore, the gastrin compounds by virtue of the presence of X, can have any of gastrin sequences from positions 18-28, 19-28, 20-28, 21-28, etc. The gastrin compound optionally contains an amino acid spacer (Y) of length m, and m is 0 to about 20 residues.

In embodiments, X is one or more amino acid residues from position 1 to 11 or 2 to 11 of SEQ ID NO: 13 or 14. Therefore, the gastrin compounds by virtue of the presence of X, can have any of gastrin sequences from positions 2 to 11, 3 to 11, 4 to 11, 5 to 11, etc. The gastrin compound optionally contains an amino acid spacer (Y) of length m, and m is 0 to about 20 residues.

A gastrin compound includes a modified gastrin compound of the formula Xₙ-AA₁-AA₂-AA₃-AA₄-AA₅-AA₆, where there is no spacer (Y) and m is 0, which may further comprise a bifunctional cross-lining agent for interaction or linkage to a carrier Z, where Z further comprises a non-proteinaceous polymer such as dextran or PEG.

A modified gastrin compound particularly described herein may further comprise an amino terminal cysteine or lysine residue.

In some embodiments of modified gastrin compounds described herein, the gastrin component contains at least amino acid residues 29-34 of SEQ ID NO: 11 or 12, and it is associated with a polymer, a lipid or a carbohydrate. The polymer may be a synthetic or naturally occurring polymer. The term polymer includes a protein polymer of amino acids, and is not limited to a synthetic polymer. The polymer may be a polyethylene glycol (PEG) or a dextran. A modified gastrin compound can be based on SEQ ID NO: 11 or 12 or “big” gastrin-34 and have a residue at position 32 which is a methionine or a leucine, respectively.

Another preferred modified gastrin compound comprises a structure C-Yₘ-X, wherein C is Cys or Lys, Yₘ is an optional spacer region comprising m amino acid residues of a small neutral amino acid, and X is at least six amino acid residues comprising at least positions 12-17 of gastrin-17 (SEQ ID NO: 13 or 14) or at least positions 29-34 of gastrin-34 (SEQ ID NO: 11 or 12). This modified gastrin compound can further comprise a bifunctional cross-linking agent wherein one reactive portion of the cross-linking agent is covalently linked to C, and the other reactive portion is covalently linked to a polymer or protein.

In a particular aspect of the invention AA₁-AA₂-AA₃-AA₄-AA₅-AA₆ in a modified gastrin compound is Tyr-Gly-Trp-Met-Asp-Phe [SEQ ID NO. 23] or Tyr-Gly-Trp-Leu-Asp-Phe [SEQ ID NO.24].

Gastrin compounds may be synthesized by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. B. Wansch, Vol. 15 I and II, Thieme, Stuttgart). The synthesis may be performed using manual procedures or by automation. Automated synthesis may be carried out, for example, using an Applied Biosystems 431A peptide synthesizer (Perkin Elmer). Gastrin compounds may also be obtained from commercial sources. For example, synthetic human gastrin 17 with methionine or leucine at position 15 are available from Bachem AG, Bubendorf, (Switzerland), and from Research Plus Inc (New Jersey, USA).

A “gastrin/CCK receptor” refers to a member of the G-protein-coupled receptor family that displays a characteristic binding affinity for a cholecystokinin (CCK) including without limitation CCK-8, desulfated CCK-8, CCK-33, CCK-4, or gastrins including without limitation desulfated or sulfated gastrin-17, or pentagastrin, or other CCK or gastrin analogues or family members. Examples of CCK/gastrin receptor proteins are CCK_{A} and CCK_{B}/gastrin receptors, in particular a CCK_{B}/gastrin receptor.

“Condition(s) and/or disease(s)” refers to one or more pathological symptoms or syndromes for which either or both a GLP-1 agonist or a gastrin compound provide a beneficial or therapeutic effect. The condition and/or disease may require reduction of blood glucose levels, inhibition of gastric acid secretion, inhibition of apoptosis of β-cells, stimulation of proliferation or differentiation of β-cells, and reduction of body weight Examples of conditions and/or diseases include but are not limited to dyslipidemia, hyperglycemia, severe hypoglycemic episodes, stroke, left ventricular hypertrophy, arrhythmia, bacteraemia, septicaemia, irritable bowel syndrome, functional dyspepsia, diabetes, catabolic changes after surgery, stress induced hyperglycemia, respiratory distress syndrome, gastric ulcers, myocardial infarction, impaired glucose tolerance, hypertension, chronic heart failure, fluid retentive states, metabolic syndrome and related diseases and disorders, obesity, diabetic complications as well as symptoms of other diseases in which tissue is damaged due to elevated glucose levels, including Alzheimer's Disease, Parkinson's Disease, and other age-related, tissue-degenerative diseases, as well as the artherogenic effects of elevated leptin, for example in patients with impaired glucose tolerance and obese non-diabetic patients.

The term, “diabetes” as used herein means any manifested symptoms of diabetes in any mammal including experimental animal models, and including human forms such as type I and type II diabetes, early stage diabetes, and a pre-diabetic condition characterized by mildly decreased insulin or mildly elevated blood glucose levels. A “pre-diabetic condition” describes a subject demonstrating a symptom in terms of insulin or glucose level, and/or demonstrating a susceptibility to diabetes or a related condition due to family history, genetic predisposition, or obesity in the case of type II diabetes, and includes a subject who has previously had diabetes or a related condition and is subject to risk of recurrence.

“Insulinotropic activity” refers to an ability of a substance to stimulate insulin secretion in response to elevated glucose levels to produce or increase glucose uptake by cells and decreased serum glucose or blood glucose levels. Methods known in the art can be employed to assay for insulinotropic activity. For example, in vitro and in vivo methods may be used that measure GLP-1 receptor binding activity or gastrin receptor binding activity, receptor activation (see the methods described in EP 619,322 to Gelfand et al and U.S. Pat. No. 5,120,712), and/or insulin or C-peptide levels. Compounds, compositions or conjugates described herein have insulinotropic activity if islet cells secrete insulin in the presence of the compounds, compositions, or conjugates above background levels or levels in the absence of the compounds, compositions, or conjugates. A compound may be administered to an animal and the insulin concentration can be monitored over time.

“Islet neogenesis” means formation of new beta cells by differentiation, which may or may not have the characteristics of stem cells which have the ability to reproduce in an unlimited manner.

### DETAILED DESCRIPTION OF EMBODIMENTS OF THE INVENTION

The invention is related to compositions, conjugates, and methods that utilize a GLP-1 agonist and a gastrin compound to provide beneficial effects. In particular, the invention relates to compositions, conjugates, and methods for the prevention, intervention and/or treatment of a condition and/or disease discussed herein comprising a GLP-1 agonist and a gastrin compound. In aspects of the invention, the compositions, conjugates and methods of the invention provide enhanced beneficial effects, in particular sustained beneficial effects relative to a GLP-1 agonist and/or a gastrin compound alone. The beneficial effects may be additive or synergistic effects.

In aspects of the invention, where the condition and/or disease is diabetes, beneficial effects, in particular sustained beneficial effects of a composition, combination treatment, or conjugate of the invention may manifest as one or more of the following:
a) An increase in pancreatic insulin levels relative to the levels measured in the absence of the active compounds or for each compound alone after administration to a subject with symptoms of diabetes. Preferably the compounds together induce at least about a 0.05%, 0.1%, 0.5%, 1%, 2%, 5%, 10%, 15%, 20%, 30%, 33%, 35%, 40%, 45%, or 50% increase in pancreatic insulin levels in a subject.
b) A reduction of an absence of symptoms of islet inflammation after administration to a subject with symptoms of diabetes.
c) A decrease in blood glucose levels relative to the levels measured in the absence of the compounds or for each compound alone in subjects with symptoms of diabetes. Preferably, the compounds induce at least about a 1%, 2%, 5%, 10%, 15%, 20%, 30%, 40%, 50%, 60%, 70%, 80%, or 90% decrease in blood glucose levels. Most preferably, the compounds yield blood glucose levels about or close to the levels common in a normal subject
d) An increase in C-peptide levels relative to the levels measured in the absence of the compounds or for each compound alone in subjects with symptoms of diabetes. Preferably, the compounds together induce at least about a 0.05%, 0.1%, 0.5%, 1%, 2%, 5%, 10%, 15%, 20%, 30%, 33%, 35%, 40%, 45%, or 50% increase in C-peptide levels.
e) Maintenance of blood glucose levels at about normal for a prolonged period of time.
f) Maintenance of hemoglobin A1c or glycated hemoglobin at about normal levels for a prolonged period of time, in particular maintaining a % hemoglobin A1c at between 6-8%, more particularly at about 7%.
g) A reduction in destruction of beta-cells. Preferably the compounds induce at least about a 1%, 2%, 5%, 10%, 15%, 20%, 30%, 40%, 50%, 60%, 70%, 80%, or 90% reduction.
h) An increase in beta-cell function. Preferably the compounds induce at least about a 1%, 2%, 5%, 10%, 15%, 20%, 30%, 40%, 50%, 60%, 70%, 80%, or 90% increase in beta-cell function.
i) A reduction, prevention, or slowing of the rate of disease progression in a subject with diabetes.
j) A reduction or prevention of the development of severe hyperglycemia and ketoacidosis with symptoms of diabetes.
k) An increase in survival in a subject with symptoms of diabetes.

In embodiments of the invention, beneficial effects or sustained beneficial effects comprise or consist essentially of two, three, four, five six, seven, eight, nine, ten, or eleven of a) through k). In particular embodiments, beneficial effects or sustained beneficial effects comprise or consist essentially of a), b), and c); a), b), c), and d); a), b), c), d), and e); a), b), c), d), e), and f); a), b), c), d), e), f), and g); a), b), c), d), e), f), g), and h); a), b), c), d), e), f), g), h), and i); a), b), c), d), e), f), g), h), i) and j); a), d), and e); a), d), e), and h); a), d), e), h), and i); a), d), e), h), i), and j); a), b), c), d), e), h), i), and j); a), b), c), d), e), h), i), j), and k); b), c), d), and e); b), c), d), e), h), i), and j); and, b), h), i) and j).

One or more of these beneficial effects or sustained beneficial effects can be demonstrated in a diabetic subject or disease model, for example a non-obese (NOD) mouse with symptoms of diabetes, using standard methods known to the skilled artisan. For example, commercially available methods and kits may be used to assay pancreatic insulin levels, glucose levels, C-peptide levels and hemoglobin A1c.

A gastrin compound may be selected for particular embodiments in the present invention and to provide a specific beneficial effect(s) based on characteristics including its insulinotrophic activity, the ability to augment the activity of a GLP-1 agonist (in particular to enhance the insulinotropic effects of a GLP-1 agonist), and/or increase the physical or chemical stability of a GLP-1 agonist. A gastrin compound can also be selected based on its ability to stimulate proliferation/differentiation of beta cells, and its in vivo half-life.

In an aspect of the invention, a gastrin compound used in the methods, compositions, and conjugates of the invention is gastrin 17 and analogs and derivatives thereof, associated with a polymer. In a particular aspect, the gastrin compound is synthetic human gastrin I having 17 amino acid residues with a Leu residue at amino acid position 15 [SEQ ID NO. 14].

In another aspect of the invention, a gastrin compound used in the methods, compositions and conjugates of the invention is gastrin 34 and analogs and derivatives thereof. In a particular aspect, the gastrin compound is a synthetic human gastrin 34 with methionine or leucine at position 32 [SEQ ID NO. 11 or 12].

In a further aspect of the invention, a gastrin compound used in the methods, compositions and conjugates of the invention is gastrin 34 or gastrin 17 or portions thereof, directly or indirectly interacting or associated with a serum protein, in particular albumin or an immunoglobulin, more particularly human serum album.

In particular aspects of the invention, a gastrin compound comprises synthetic human gastrin 34 having 2-34 amino acid residues of SEQ ID NO. 11 or 12, and optionally an N-terminal cysteine and/or a carrier; synthetic human gastrin having 1-17 amino acid residues with a Leu residue at amino acid position 15 [SEQ ID NO. 14] and optionally an N-terminal cysteine residue; and a synthetic human gastrin having amino acid residues 2 to 17 or 5-17 of SEQ ID NO. 13 or 14, optionally with an N-terminal cysteine residue and/or a carrier (e.g. PEG or human serum albumin) linked via a spacer [e.g. Gly-Ala-Gly-Ala-Gly-Ala-Gly-Ala-Gly-Ala i.e. (GA)₅] [SEQ ID NO. 25], in particular, a synthetic human gastrin having amino acid residues 2 to 17 or 5-17 of SEQ ID NO. 13 or 14, with a human serum albumin (HSA) polymer linked via a Gly-Ala-Gly-Ala-Gly-Ala-Gly-Ala-Gly-Ala [ie. (GA)₅] spacer, and optionally an N-terminal cysteine residue.

A GLP-1 agonist may be selected for particular applications in the present invention based on one or more of the following characteristics: ability to bind to the GLP-1 receptor, preferably with an affinity constant K_{d} less than about 1 μM, more preferably less than about 100 nM; ability to initiate a signal transduction pathway resulting in insulinotropic activity; insulinotropic activity; stimulation of beta cell proliferation/differentiation; resistance to DP IV cleavage; and, an in vivo half-life of at least about 15 minutes to 24 hours, preferably 2 to 10 hours or 2 to 8 hours in humans using conventional methods (see for example, the method described in US 2003/0144206)

In aspects of the invention the GLP-1 agonist is a naturally truncated GLP-1 polypeptide (GLP-1(7-36) or ((GLP-1(7-37)), or an analogue or derivative thereof. The sequences of these naturally occurring truncated GLP-1 agonists are represented in SEQ ID NOs. 4, 5, and 6.

In certain aspects of the invention, a GLP-1 agonist may have the amino acid sequence of SEQ ID NOs. 1, 2, or 3 modified so that amino acid residues at positions 1-20, preferably 1-15, more preferably 1-10, most preferably 1-5 differ from the sequences of SEQ ID NOs. 1, 2 or 3.

In an embodiment of the invention, the GLP-1 agonist is an analogue of GLP-1(7-37) or GLP-1(7-36) which has less than 10 amino acid residues that are different from those in GLP-1(7-37) or GLP-1(7-36), less than 5 amino acid residues that are different from those in GLP-1(7-37) or GLP-1(7-36), less than 3 amino acid residues that are different from those in GLP-1 (7-37) or GLP-1(7-36), preferably only one amino acid residue that is different from sequence of GLP-1(7-37) or GLP-1(7-36).

GLP-1 agonists that may have specific utility in the present invention include polypeptides where one or more amino acids have been added to the N-terminus and/or C-terminus of GLP-1(7-37) or GLP-1(7-36). Preferably, about one to six amino acids may be added to the N-terminus and/or from about one to eight amino acids may be added to the C-terminus. In certain applications GLP-1 agonists are selected that have up to 39 amino acid. Amino acids at positions 1-6 of an extended GLP-1 agonist may be selected so that they are the same or are conservative substitutions of the amino acid at the corresponding positions of the parent GLP-1(7-37) or GLP-1(7-36). Amino acids at positions 38-45 of an extended GLP-1 agonist may be selected so that they are the same or conservative substitutions of the amino acids at the corresponding positions of exendin-3 or exendin-4 (SEQ ID NO. 7 and 8 respectively).

In aspects of the invention a GLP-1 agonist is utilized comprising a position 8 analogue wherein the backbone for such analogs or fragments thereof contain an amino acid other than alanine. The amino acid at position 8 may be selected from glycine, valine, leucine, isoleucine, serine, threonine, or methionine.

In an embodiment a GLP-1 agonist is an insulinotropic analogue of GLP-1(1-37), for example, Met⁸-GLP-1(7-37), wherein the alanine in position 8 has been replaced by methionine and the amino acid residues in position 1 to 6 have been deleted, and Arg³⁴-GLP-1(7-37) wherein the valine in position 34 has been replaced with arginine and the amino acid residues in position 1 to 6 have been deleted.

In another embodiment, LP-1 agonists are selected that have the sequence GLP-1(7-37)OH and GLP-1(7-36) amide, and the corresponding position 8 analogs wherein the backbone for such analogs contains an amino acid other than alanine. The amino acid at position 8 may be selected from glycine, valine, leucine, isoleucine, serine, threonine, or methionine, preferably valine or glycine. The analogs may additionally contain (a) an amino acid at position 22 selected from glutamic acid, lysine, aspartic acid, arginine, and preferably glutamic acid or lysine; (b) an amino acid at position 30 selected from glutamic acid, aspartic acid, serine, or histidine; (c) an amino acid at position 37 selected from lysine, arginine, threonine, glutamic acid, aspartic acid, serine, tryptophan, tyrosine, phenylalanine, or histidine; and/or (d) amino acid at position 27 selected from alanine, lysine, arginine, tryptophan, tyrosine, phenylalanine, or histidine.

A group of GLP-1 analogs and derivatives for use in the present invention comprises the GLP-1 agonists described in U.S. Pat. No. 5,545,618 and US Patent Application Serial No. 20040018975. The analogs include active GLP-1 peptides, 7-34, 7-35, 7-36 and 7-37 having amino acid substitutions at positions 7-10 and/or are truncations at the C-terminus and/or contain various other amino acid substitutions in the basic peptide. Preferred analogs include those with D-amino acid substitutions in the 7 and 8 positions and/or N-alkylated or N-acylated amino acids in the 7 position since they are particularly resistant to degradation in vivo.

In aspects of the invention, a GLP-1 agonist comprises a peptide comprising or selected from the group consisting of GLP-1 (1-38); GLP-1 (1-39), GLP-1 (1-40), GLP-1 (1-41), GLP-1 (7-38), GLP-1 (7-39), GLP-1 (7-40), and GLP-1 (7-41).

In another aspect of the invention at least one amino acid of a GLP-1 agonist has at least one substituent attached directly or indirectly (e.g. via a spacer such as γ-Glu or β-Ala). A substituent is generally selected to make the profile of action of the parent GLP-1 agonist more protracted, make the GLP-1 agonists more metabolically and physically stable, and/or increase solubility of the GLP-1 agonist. An example of a particular substituent is an amide, a carbohydrate, and a lipophilic substituent. A lipophilic substituent includes but is not limited to an alkyl group, a group which has an ω-carboxylic acid group, an acyl group of a straight-chain or branched fatty acid or alkane such as tetradecanoyl, hexadecanoyl. Particular compositions, conjugates and treatments of the invention use GLP-1 agonists with lipophilic substitutents such as those described in WO 99/43341 (Novo Nordisk) and US 2003/0119734A1 (Novo Nordisk).

In particular aspects of the invention a GLP-1 agonist is a GLP-1(7-36)-amide or Tyr³¹-exendin-4(1-31)-amide.

Certain aspects of the invention provide a GLP-1 agonist that is a derivative of GLP-1 (7-36) or GLP-1 (7-37) comprising a lipophilic substitutent. In an embodiment, the GLP-1 agonist is Arg³⁴Lys²⁶(N^{ε}(γ-Glu(N^{α}-hexadecanoyl)))-GLP-1(7-37).

In embodiments of the invention, the GLP-1 agonist comprises or is selected from the group consisting of: Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37)-Val⁸Arg²²-GLP-1(7-36)-amide, Val⁸Arg²²-GLP-1(7-37), Val⁸His²²-GLP-1(7-36)-amide, Val⁸His²²-GLP-1(7-37), Arg²⁶-GLP-1(7-37); Arg³⁴-GLP-1(7-37); Lys³⁶-GLP-1(7-37); Arg^{26,34}Lys³⁶-GLP-1(7-37); Arg^{26,34}-GLP-1(7-37); Arg^{26,34}Lys⁴⁰-GLP-1(7-37); Arg²⁶Lys³⁶-GLP-1(7-37); Arg³⁴Lys³⁶-GLP-1(7-37); Val⁸Arg²²-GLP-1(7-37); Met⁸Arg²²-GLP-1(7-37); Gly⁸His²²-GLP-1(7-37); Val⁵His²²-GLP-1(7-37); Met⁸His²²-GLP-1(7-37); His³⁷-GLP-1(7-37); Gly⁸-GLP-1(7-37); Val⁸-GLP-1(7-37); Met⁸-GLP-1(7-37); Gly⁸Asp²²-GLP-1(7-37); Val⁸Asp²²-GLP-1(7-37); Met⁸Asp²²-GLP-1(7-37); Gly⁸Glu²²-GLP-1(7-37); Val⁸-GLP-1(7-37); Gly⁸Lys²²-GLP-1(7-37); Val⁸Lys²²-GLP-1(7-37); Glu²²Met⁸Glu²²-GLP-1(7-37); Gly⁸Lys²²-GLP-1(7-37); Val⁸Lys²²-GLP-1(7-37); Met⁸Lys²²-GLP-1(7-37); Gly⁸Arg²²-GLP-1(7-37); Val⁸Lys²²His³⁷-GLP-1(7-37); Gly⁸Glu²²His³⁷-GLP-1(7-37); Val⁸Glu²²His¹⁷-GLP-1(7-37); Met⁸Glu²²His³⁷-GLP-1(7-37); Gly⁸Lys²² His³⁷-GLP-1(7-37); Met⁸Lys²²His³⁷-GLP-1(7-37); Gly⁸Arg²²His³⁷-GLP-1(7-37); Val⁸Arg²²His³⁷-GLP-1(7-37); Met⁸Arg²²His³⁷ GLP-1(7-37); Gly⁸His²²His³⁷-GLP-1(7-37); Val⁸His²²His³⁷-GLP-1(7-37); Met⁸His²²His³⁷-GLP-1(7-37); Gly⁸His³⁷-GLP-1(7-37); Val⁸His³⁷-GLP-1(7-37); Met⁸His³⁷-GLP-1(7-37); Gly⁸Asp²²His³⁷-GLP-1(7-37); Val⁸Asp²²His³⁷-GLP-1(7-37); Met⁸Asp²²His³⁷-GLP-1(7-37); Arg²⁶-GLP-1(7-36)-amide; Arg³⁴-GLP-1(7-36)-amide; Lys³⁶-GLP-1(7-36)-amide; Arg^{26,34}Lys³⁶-GLP-1-(7-36)-amide; Arg^{26,34}-GLP-1(7-36)-amide; Arg^{26,34}Lys⁴⁰-GLP-1(7-36)-amide; Arg²²Lys³⁶-GLP-1(7-36)-amide; Arg³⁴Lys³⁶-GLP-1(7-36)-amide; Gly⁸-GLP-1(7-36)-amide; Val⁸-GLP-1(7-36)-amide; Met⁸-GLP-1(7-36)-amide; Gly⁸Asp²²-GLP-1(7-36)-amide; Gly⁸Glu²²His³⁷-GLP-1(7-36)-amide; Val⁸Asp²²-GLP-1(7-36)-amide; Met⁸Asp²²-GLP-1(7-36)-amide; Gly⁸Glu²²-GLP-1(7-36)-amide; Val⁸Glu²²-GLP-1(7-36)-amide; Met⁸Glu²²-GLP-1(7-36)-amide; Gly⁸Lys²²-GLP-1(7-36)-amide; Val⁸Lys²²-GLP-1(7-36)-amide; Met⁸Lys²²-GLP-1(7-36)-amide; Gly⁸His²²His³⁷-GLP-1(7-36)-amide; Gly⁸Arg²²-GLP-1(7-36)-amide; Val⁸Arg²²-GLP-1(7-36)-amide; Met⁸Arg²²-GLP-1(7-36)-amide Met⁸Arg²²His³⁷-GLP-1(7-36)-amide; Gly⁸His²²-GLP-1(7-36)-amide; Val⁸His²²-GLP-1(7-36)-amide; Met⁸His²²-GLP-1(7-36)-amide; His³⁷-GLP-1(7-36)-amide; Val⁸Arg²²His³⁷-GLP-1(7-36)-amide; Met⁸Arg²²His³⁷-GLP-1(7-36)-amide; Gly⁸His³⁷-GLP-1(7-36)-amide; Val⁸His³⁷-GLP-1(7-36)-amide; Met⁸His³⁷-GLP-1(7-36)-amide; Gly⁸Asp²²His³⁷-GLP-1(7-36)-amide; Val⁸Asp²²His³⁷-C-GLP-1(7-36)-amide; Met⁸Asp²²His³⁷-GLP-1(7-36)-amide; Val⁸Glu²²His³⁷-GLP-1(7-36)-amide; Met⁸Glu²²His³⁷-GLP-1(7-36)-amide; Gly⁸Lys²²His³⁷-GLP-1(7-36)-amide; Val⁸Lys²²His³⁷-GLP-1(7-36)-amide; Met⁸Lys²²His³⁷-GLP-1(7-36)-amide; Gly⁸Arg²²His³⁷-GLP-1(7-36)-amide; Val⁸His²²His³⁷-GLP-1(7-36)-amide; Met⁸His²²His³⁷-GLP-1(7-36)-amide; Val⁸-GLP-1(7-37)OH, Gly⁸-GLP-1(7-37)OH, Glu-GLP-1(7-37)OH, Asp²²-GLP-1(7-37)OH, Arg²²-GLP-1(7-37)OH, Lys²²-GLP-1(7-37)OH, Cys²²-GLP-1(7-37)OH, Val⁸-Glu²²GLP-1(7-37)OH, Val⁸-Asp²²-GLP-1(7-37)OH, Val⁸-Arg²²-GLP-1(7-37)OH, Val⁸-Lys²²-GLP-1(7-37)OH, Val⁸-Cys²²-GLP-1(7-37)OH, Gly⁸-Glu²²-GLP-1(7-37)OH, Gly⁸-Asp²²-GLP-1(7-37)OH, Gly⁸-Arg²²-GLP-1(7-37)OH, Gly⁸-Lys²²-GLP-1(7-37)OH, Gly⁸-Cya²²-GLP-1(7-37)OH, Glu²²-GLP-1(7-36), NH₂, ASP²²-GLP-1(7-36)NH₂, Arg²²-GLP-1(7-36)NH₂, Lys²²-GLP-1(7-36)NH₂, Cys²²-GLP-1(7-36)NH₂, Val⁸-Glu²²-GLP-1(7-36)NH₂, Val⁸-Asp²²-GLP-1(7-36)NH₂, Val⁸-Arg²²-GLP-1(7-36)NH₂, Val⁸-Lys²²-GLP-1(7-36)NH₂, Val⁸-Cys²²-GLP-1(7-36)NH, Gly⁸-Glu-GLP-1(7-36)NH, Gly⁸-Asp-GLP-1(7-36)NH₂, Gly⁸-Arg²²-GLP-1(7-36)NH₂, Gly⁸-Lys²²-GLP-1(7-36)NH₂, Gly⁸-Cys²²-GLP-1(7-36)NH₂, Lys23-GLP-1(7-37)OH, Val⁸-Lys²⁴-GLP-1(7-37)OH, Gly⁸-Lys²³-GLP-1(7-37)OH, His²⁴-GLP-1 (7-37)OH, Val⁸-His²⁴-GLP-1(7-37)OH, Gly⁸-His²⁴-GLP-1(7-37)OH, Lys²⁴-GLP-1(7-37)OH, Val⁸-Lys²²-GLP-1(7-37)OH, Gly⁸-Lys²³-GLP-1(7-37)OH, Glu³⁰-GLP-1(7-37)OH, Val⁸Glu³⁰-GLP-1(7-37)OH, Gly⁸-Glu³⁰-GLP-1(7-37)OH, Asp³⁰-GLP-1(7-37)OH, Val⁸-Asp³⁰-GLP-1(7-37)OH, Gly⁸-Asp³⁰-GLP-1(7-37)OH, Gln³⁰-GLP-1(7-37)OH, Val⁸-Gln³⁰-GLP-1(7-37)OH, Gly⁸-Gln³⁰GLP-1(7-37)OH, Tyr³⁰-GLP-1(7-37)OH, Val⁸-Tyr³⁰-GLP-1(7-37)OH, Gly⁸-Tyr³⁰-GLP-1(7-37)OH, Ser³⁰-GLP-1(7-37)OH, Val⁸-Ser³⁰-GLP-1(7-37)OH, Gly⁸-Ser³⁰-GLP-1(7-37)OH, His³⁰-GLP-1(7-37)OH, Val⁸-His³⁰-GLP-1(7-37)OH, Gly⁸-His³⁰-GLP-1(7-37)OH, Glu³⁴-GLP-1(7-37)OH, Val⁸-Glu³⁴-GLP-1(7-37)OH, Gly⁸-Glu³⁴-GLP-1(7-37)OH, Ala³⁴-GLP-1(7-37)OH, Val⁸-Ala³⁴-GLP-1(7-37)OH, Gly⁸-Ala³⁴-GLP-1(7-37)OH, Gly³⁴-GLP-1(7-37)OH, Val⁸-Glu³⁴-GLP-1(7-37)OH, Gly⁸-Gly³⁴-GLP-1(7-37)OH, Ala³⁵GLP-1(7-37)OH, Val⁸-Ala³⁰S-GLP-1(7-37)OH, Gly⁸-Ala³⁵-GLP-1(7-37)OH, Lys³⁵-GLP-1(7-37)OH, Val⁸-Lys³⁵-GLP-1(7-37)OH, Gly⁸-Lys³⁰-GLP-1(7-37)OH, His³⁵-GLP-1(7-37)OH Val⁸-His³⁵-GLP-1(7-37)OH, Gly⁸-His³⁰-GLP-1(7-37)OH, Pro³⁵-GLP-1(7-37)OH Val⁸-Pro³⁵-GLP-1(7-37)OH, Gly⁸-Pro³⁵-GLP-1(7-37)OH, Glu³⁵-GLP-1(7-37)OH, Val⁸-Glu³⁵-GLP-1(7-37)OH, Glu⁸-Glu³⁵-GLP-1(7-37)OH, Val⁸-Ala²⁷-GLP-1(7-37)OH, Val⁸-His³⁷-GLP-1(7-37)OH, Val⁸-Glu²²-Lys²³-GLP-1(7-37)OH, Val⁸-Glu²²-Glu²³-GLP-1(7-37)OH, Val⁸-Glu²²-Ala²⁷-GLP-1(7-37)OH, Val⁸-Gly³⁴-Lys³⁵-GLP-1(7-37)OH, Val⁸-His³⁷-GLP-1(7-37)OH, and Gly⁸-His³⁷-GLP-1(7-37)OH, and derivatives thereof.

In a particular embodiment a GLP-1 agonist comprises or is selected from the group consisting of Val⁸-GLP-1(7-37)OH, Gly⁸-GLP-1(7-37)OH, Glu²²-GLP-1(7-37)OH, Lys²²-GLP-1(7-37)OH, Val⁸-Glu²²-GLP-1(7-37)OH, Val⁸-Lys²²-GLP-1(7-37)OH, Gly⁸-Glu²²-GLP-1(7-37)OH, Gly⁸-Lys²²-GLP-1(7-37)OH, Glu-GLP-1(7-36)NH₂, Lys²²-GLP-1(7-36)NH₂, Val⁸-Glu²²-GLP-1(7-36)NH₂, Val⁸-Lys²²-GLP-1(7-36)NH₂, Gly⁸-Glu²²-GLP-1(7-36)NH₂, Gly⁸-Lys²²-GLP-1(7-36)NH, Val⁸-His³⁷-GLP-1(7-37)OH, Gly⁸-His³⁷-GLP-1(7-37)OH, Arg³⁴-GLP-1(7-36)NH₂, and Arg³⁴-GLP-1(7-37)OH.

In another particular embodiment, the GLP-1 agonist comprises or is selected from the group consisting of Gly⁸-GLP-1(7-37), Val⁸GLP-1(7-37), Val⁸Asp²²GLP-1(7-37), Val⁸Glu²²GLP-1(7-37), Val⁸Lys²²GLP-1(7-37), and Val⁸His²²GLP-1(7-37), and analogs and derivatives thereof.

In a further particular embodiment, the GLP-1 agonist comprises or is selected from the group consisting of Gly⁸-GLP-1(7-36) amide, Val⁶GLP-1(7-36) amide, Val⁸Asp²²GLP-1(7-36) amide, Val⁸Glu²²GLP-1(7-36) amide, Val⁸Lys²²GLP-1(7-36) amide, and Val⁸His²²GLP-1(7-36) amide, and analogs and derivatives thereof.

In still further embodiments of the invention, a GLP-1 agonist is exendin (e.g. exendin 3 and exendin 4) or an analog, derivative, or fragment thereof. Examples of exendins that may be utilized in the present invention include without limitation those disclosed in WO 9746584, U.S. Pat. No. 5,424,286 and WO 01/04156. U.S. Pat. No. 5,424,286 describes use of an exendin polypeptide for stimulating insulin release. The exendin polypeptides described in the US patent include HGEOTFRSDLSKQMEEBAVRLFIBWLKNGGX wherein X=P or Y, and HX₁X²GTFITSDLSKQMEBBAVRLPIEWLKNGGPSSGAPPPS; wherein X¹X²¹ SD (exendin-3) or GB (exendin-4). WO 9746584 describes truncated exendin peptides that increase secretion and biosynthesis of insulin but reduce glucagons. WO 01/04156 describes analogs and derivatives of exendin-4 and their preparation.

In another embodiment, a GLP-1 agonist is an insulinotropic analogue of exendin-4(1-39), in particular Ser²Asp³-exendin-4(1-39Y wherein the amino acid residues in position 2 and 3 have been replaced with serine and aspartic acid, respectively (this particular analogue is also being known in the art as exendin-3, SEQ ID NO. 7).

In certain aspects of the invention the GLP-1 agonist is a stable GLP-1 agonist in particular a stable GLP-1 analogue or derivative, or a stable exendin-4 or exendin-3 analogue or derivative.

Pharmaceutical compositions of the invention can be selected that provide beneficial effects, in particular statistically significant beneficial effects or sustained beneficial effects, compared with a GLP-1 agonist or a gastrin compound alone. Beneficial effects in respect to a diabetic condition may be evidenced by one or more of the beneficial effects described herein, in particular one, two, three, four, five, six, seven, eight, nine or ten of the beneficial effects described above in a) through j).

A pharmaceutical composition with beneficial effects, in particular statistically significant beneficial effects or sustained beneficial effects, is provided comprising a GLP-1 agonist selected from the group consisting of Gly⁸-GLP-1(7-37), Val⁸GLP-1(7-37), Val⁸Asp²²GLP-1(7-37), Val⁸Glu²²GLP-1(7-37), Val⁸Lys²²GLP-1(7-37), Val⁸His²²GLP-1(7-37), Arg³⁴Lys²⁶(N^{ε}(γ-Glu(N^{α}-hexadecanoyl)))-GLP-1(7-37), Gly⁸-GLP-1(7-36) amide, Val⁸GLP-1(7-36) amide, Val⁸Asp²²GLP-1(7-36) amide, Val⁸Glu²²GLP-1(7-36) amide, Val⁸Lys²²GLP-1(7-36) amide, and Val⁸His²²GLP-1(7-36) amide, and a gastrin compound (e.g. SEQ ID NO. 11, 12, 13 or 14), optionally associated with a serum protein.

In an aspect, a pharmaceutical composition with statistically significant beneficial effects or sustained beneficial effects is provided comprising GLP-1(7-36) [SEQ ID NO. 5] and gastrin-17(leu) [SEQ ID NO.14].

In an aspect, a pharmaceutical composition with statistically significant beneficial effects or sustained beneficial effects is provided comprising Arg³⁴Lys²⁶(N^{ε}(γ-Glu(N^{α}-hexadecanoyl)-GLP-1(7-37), and gastrin-17(leu) [SEQ ID NO.14].

In another aspect, a pharmaceutical composition with statistically significant beneficial effects or sustained beneficial effects is provided comprising Aib^{8,35} GLP-1(7-36) amide or Ser³⁸,Lys^{39,40,41,42,43,44}-Exendin-4(1-39)amide and gastrin-17(leu) [SEQ ID NO.14].

A pharmaceutical composition with beneficial effects, in particular statistically significant beneficial effects or sustained beneficial effects, is provided comprising a GLP-1 agonist selected from the group consisting of Gly⁸-GLP-1(7-37), Val⁸GLP-1(7-37), Val⁸Asp³²GLP-1(7-37), Val⁸Glu²²GLP-1(7-37), Val⁸Lys²²GLP-1(7-37), Val⁸His²²GLP-1(7-37), Arg³⁴Lys²⁶(N^{ε}(γ-Glu(N^{α}-hexadecanoyl)))-GLP-1(7-37), Gly⁸-GLP-1(7-36) amide, Val⁸GLP-1(7-36) amide, Val⁸Asp²²GLP-1(7-36) amide, Val⁸Glu²²GLP-1(7-36) amide, Val⁸Lys²²GLP-1(7-36) amide, and Val⁸His²²GLP-1(7-36) amide, and a gastrin compound having an amino acid sequence comprising, from the amino terminus, Z-Yₘ-Xₙ-AA₁-AA₂-AA₃-AA₄-AA₅-AA₆, wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr, Z is a polymer and when the polymer is a protein Z is an amino acid sequence; Yₘ is an optional spacer region comprising m amino acid residues of a small neutral amino acid including but not limited to serine and alanine, and X is any consecutive portion of residues 1-28 of SEQ ID NO: 11 or 12, or residues 1-11 of SEQ ID NO. 13 or 14, preferably AA₁-AA₂-AA₃-AA₄-AA₅-AA₆ is Tyr-Gly-Trp-Met-Asp-Phe or Tyr-Gly-Trp-Leu-Asp-Phe. In a particular embodiment, Z is a serum protein, in particular human serum albumin.

In certain aspects of the invention, pharmaceutically acceptable salts of a GLP-1 agonist and/or pharmaceutically acceptable salts of a gastrin compound are utilized.

The invention in particular aspects provides a pharmaceutical composition which has been adapted for administration to a subject to provide sustained beneficial effects to treat a condition and/or disease, preferably diabetes. In an embodiment for the prevention and/or treatment of diabetes, the composition is in a form such that administration to a subject results in blood glucose levels that are about normal that persist in the subject for a prolonged period of time after cessation of treatment.

This invention provides a conjugate comprising a GLP-1 agonist linked to or interacting with a gastrin compound wherein the interaction is for example, via an amino or a carboxyl group. The invention also relates to isolated covalent conjugates of the invention, and compositions comprising covalent conjugates of the invention. A GLP-1 agonist may be conjugated to a species via an ester bond between an OH and a COOH of a gastrin compound. Conjugates of a GLP-1 agonist and a gastrin compound may be conjugated with an intermediate spacer or linker. A suitable spacer or linker may be a mono- or disaccharide, an amino acid, a sulfate, a succinate, an acetate, or an oligomeric polymeric spacer or linker comprising one or more of such moieties.

The invention also provides methods of preparing conjugates that result in conjugates with improved pharmacokinetic properties, biological activity, and beneficial effects. The methods comprise incubating the GLP-1 agonist with a gastrin compound under conditions that allow formation of a covalent linkage between the two compounds. The invention therefore contemplates a process for preparing a covalent conjugate comprising a GLP-1 agonist covalently bonded or linked to a gastrin compound, the process comprising: incubating the GLP-1 agonist with a gastrin compound under conditions and at a pH and for a time sufficient for formation of a covalent bond or linkage between the GLP-1 agonist and gastrin compound; and isolating the covalent conjugate. The above process for preparing a conjugate comprising a GLP-1 agonist and a gastrin compound provides a conjugate with a substantial amount of a GLP-1 agonist covalently linked to the GLP-1 agonist.

N-terminal or C-terminal fusion proteins or chimeric proteins, comprising a GLP-1 agonist conjugated with a gastrin compound, optionally with a spacer or linker, may also be prepared by fusing, through recombinant techniques, the N-terminal or C-terminal sequence of a GLP-1 agonist and the sequence of a gastrin compound.

The invention relates to a conjugate prepared by a process described herein. The invention also relates to pharmaceutical formulation or composition comprising conjugates of the invention and a pharmaceutically acceptable carrier, excipient, or vehicle.

The invention further relates to a pharmaceutical formulation or composition of substantially pure covalent conjugates comprising a GLP-1 agonist covalently linked to a gastrin compound which provides beneficial effects preferably sustained beneficial effects compared to the GLP-1 agonist alone. In an embodiment, a pharmaceutical formulation is provided consisting essentially of covalent conjugates comprising a GLP-1 agonist covalently linked without an intermediate spacer or linker to a gastrin compound. In another embodiment, a pharmaceutical formulation is provided consisting essentially of covalent conjugates comprising a GLP-1 agonist covalently linked with an intermediate spacer or linker to a gastrin compound

In aspects of the invention, a composition or conjugate comprising a GLP-1 agonist and a gastrin compound have greater sustained insulinotropic activity following treatment compared with the activity of a GLP-1 agonist or gastrin compound alone or the activity of GLP-1(7-37)OH.

The invention provides methods for the prevention, treatment and/or intervention of a condition and/or disease in a subject comprising administering a gastrin compound and a GLP-1 agonist or a pharmaceutical composition of the invention to provide a beneficial effect, in particular a sustained beneficial effect.

In methods of the invention providing beneficial effects, in particular statistically significant beneficial effects or sustained beneficial effects, a GLP-1 agonist can be selected from the group consisting of Gly⁸-GLP-1(7-37), Val⁸GLP-1(7-37), Val⁸Asp²²GLP-1(7-37), Val⁸Glu²²GLP-1(7-37), Val⁸Lys²²GLP-1(7-37), Val⁸His²²GLP-1(7-37), Arg³⁴Lys²⁶(N^{ε}(γ-Glu(N^{α}-hexadecanoyl))-GLP-1(7-37), Gly⁸-GLP-1(7-36) amide, Val⁸GLP-1(7-36) amide, Val⁸Asp²²GLP-1(7-36) amide, Val⁸Glu²²GLP-1(7-36) amide, Val⁸Lys²²GLP-1(7-36) amide, and Val⁸His²²GLP-1(7-36) amide, and a gastrin compound can comprise SEQ ID NO. 11, 12, 13 or 14, optionally associated with a serum protein.

In certain methods of the invention GLP-1(7-36) [SEQ ID NO. 5] and gastrin-17(leu) [SEQ ID NO.14] are administered.

In certain other methods of the invention Arg³⁴Lys²⁶(N^{ε}(γ-Glu(N^{α}-hexadecanoyl)GLP-1(7-37), and gastrin-17(leu) [SEQ ID NO.14] are administered.

In certain other methods of the invention Aib^{8,35} GLP-1(7-36) amide or Ser³⁸,Lys^{39,40,41,42,44}-Exendin-4(1-39)amide and gastrin-17(leu) [SEQ ID NO.14] are administered.

In certain further methods of the invention providing beneficial effects, in particular statistically significant beneficial effects or sustained beneficial effects, a GLP-1 agonist is selected from the group consisting of Gly⁸-GLP-1(7-37), Val⁸GLP-1(7-37), Val⁸Asp²²GLP-1(7-37), Val⁸Glu²²GLP-1(7-37), Val⁸Lys²²GLP-1(7-37), Val⁸His²²GLP-1(7-37), Arg³⁷Lys²⁶(N^{ε}-Glu(N^{α}-hexadecanoyl)))-GLP-1(7-37), Gly⁸-GLP-1(7-36) amide, Val⁸GLP-1(7-36) amide, Val⁸Asp²²GLP-1(7-36) amide, Val⁸Glu²²GLP-(7-36) amide, Val⁸Lys²²GLP-1(7-36) amide, and Val⁸His²²GLP-1(7-36) amide, and a gastrin compound comprises an amino acid sequence comprising, from the amino terminus, Z-Yₘ-Xₙ-AA₁-AA₂-AA₃-AA₄-AA₅-AA₆ wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr; Z is a polymer and when the polymer is a protein Z is an amino acid sequence; Yₘ is an optional spacer region comprising m amino acid residues of a small neutral amino acid including but not limited to serine and alanine, and X is any consecutive portion of residues 1-28 of SEQ ID NO: 11 or 12, or residues 1-17 of SEQ ID NO. 13 or 14, preferably AA₁-AA₂-AA₃-AA₄-AA₅-AA₆ is Tyr-Gly-Trp-Met-Asp-Phe or Tyr-Gly-Trp-Leu-Asp Phe. In a particular embodiment, Z is a serum protein, in particular human serum albumin.

In an aspect, the invention provides a method for the prevention and/or intervention of a condition and/or disease discussed herein in a subject comprising administration of at least one GLP-1 agonist and at least one gastrin compound. A GLP-1 agonist and a gastrin compound may be directly administered to a subject or contacted with cells (e.g. stem cells or progenitor cells) and administered to a subject.

The invention also provides a combination treatment for preventing and/or treating a condition and/or disease discussed herein in a subject comprising administering to the subject a therapeutically effective amount of at least one GLP-1 agonist and a gastrin compound to provide beneficial effects. In an aspect the invention provides a combination treatment or intervention which provides sustained beneficial effects following treatment.

In particular, the invention provides a combination treatment for treating or preventing a condition and/or disease in a subject comprising administering to the subject a therapeutically effective amount of at least one GLP-1 agonist and at least one gastrin compound to produce beneficial effects, preferably sustained beneficial effects.

The invention also relates to a method of treatment comprising administering a therapeutically effective amount of at least one GLP-1 agonist in combination with the administration of at least one gastrin compound which upon administration to a subject with symptoms of diabetes produces beneficial effects, preferably sustained beneficial effects, manifested as reduced blood glucose levels and/or increased pancreatic insulin.

In an aspect of the invention therapeutically effective amounts of a GLP-1 agonist and a gastrin compound are combined prior to administration to a subject. In an embodiment, therapeutically effective amounts of a GLP-1 agonist and a gastrin compound are mixed at a physiologically acceptable pH.

In an embodiment, the invention provides a method for stimulating beta cell proliferation in a subject comprising administering a therapeutically effective amount of a composition or conjugate of the invention, or administering in combination a GLP-1 agonist and a gastrin compound.

In another embodiment, the invention provides a method for increasing the number and/or size of beta cells in a subject comprising administering a therapeutically effective amount of a composition or conjugate of the invention or administering in combination a GLP-1 agonist and a gastrin compound.

In a further embodiment, the invention provides a method for preventing or treating Type I or Type II diabetes comprising administering a therapeutically effective amount of a composition or conjugate of the invention, or administering in combination a GLP-1 agonist and a gastrin compound.

In a still further embodiment, the invention provides a method for ameliorating progression of disease or obtaining a less severe stage of disease in a person suffering from Type II diabetes comprising administering a therapeutically effective amount of a composition or conjugate of the invention, or administering in combination a GLP-1 agonist and a gastrin compound.

The invention relates to a method of delaying the progression of impaired glucose tolerance or non-insulin requiring Type II diabetes to insulin requiring Type II diabetes comprising administering a therapeutically effective amount of a composition or conjugate of the invention, or administering in combination a GLP-1 agonist and a gastrin compound.

The invention also relates to a method of increasing the insulin synthesis capability of a subject comprising administering a therapeutically effective amount of a composition or conjugate of the invention, or administering in combination a GLP-1 agonist and a gastrin compound.

The invention further relates to inducing islet neogenesis in a subject comprising contacting islet precursor cells with a GLP-1 agonist and a gastrin compound, composition, or conjugate of the invention in a sufficient amount to increase proliferation of islet precursor cells in the subject thereby inducing islet neogenesis.

The invention contemplates a method of expanding a functional beta cell mass of pancreatic islet transplants in a diabetic patient, the method comprising administering to the patient a therapeutically effective amount of a GLP-1 agonist and a gastrin compound, or a composition or conjugate of the invention.

In an aspect, the invention provides methods for treating diabetes mellitus in a patient in need thereof by administering a composition comprising a gastrin compound and a GLP-1 agonist in an amount sufficient to effect differentiation of the patient's pancreatic islet precursor cells to mature insulin-secreting cells and/or to stimulate insulin synthesis in existing islet cells. The composition can be administered systemically or expressed in situ by host cells containing one or more nucleic acid construct in an expression vector wherein the nucleic acid construct comprises a coding sequence for a gastrin compound or a coding sequence for a GLP-1 agonist or for both compounds, together with transcriptional and translational regulatory regions functional in pancreatic islet precursor cells.

The invention provides methods for treating cells, preferably cells in culture using a GLP-1 agonist and gastrin compound of the invention, or compositions, or conjugates of the invention. The invention also provides cell based treatment methods using a GLP-1 agonist and a gastrin compound of the invention, or compositions, or conjugates of the invention. See PCT/CA03/33595 for a description of general culture and cell based treatment methods.

In an aspect, the invention relates to a method for expanding and differentiating stem cells or progenitor cells into insulin secreting cells comprising contacting the stem cells or progenitor cells with a GLP-1 agonist and a gastrin compound or a composition or conjugate of the invention in sufficient amounts to expand and differentiate stem cells or progenitor cells. The amount of expansion and differentiation may be significantly different compared with that achieved in the absence of the compounds, composition or conjugate, in particular the amount may be significantly greater compared with an amount achieved with a GLP-1 agonist or a gastrin compound alone. In an embodiment, the stem cells or progenitor cells are contacted with the compounds, composition, or conjugate in culture. In another embodiment, the stem cells or progenitor cells are contacted with the compounds, composition, or conjugate in a subject. The compounds, composition or conjugate may be administered to a subject before, during, or after implantation of stem cells in the subject to expand and differentiate the stem cells in the subject. The stem cells may be obtained from pancreatic islets, umbilical cords, embryos, or stem cell lines. The method may additionally comprise administering an immunosuppressive agent.

The invention also relates to a method for enhancing proliferation of insulin secreting cells in culture comprising contacting the cells with a GLP-1 agonist and a gastrin compound, composition or conjugate of the invention in sufficient amounts to enhance proliferation of the cells. The amount of proliferation may be significantly different compared with that achieved in the absence of the compounds, composition or conjugate. In an embodiment, the amount of proliferation is significantly greater compared with a GLP-1 agonist or a gastrin compound alone

The invention further relates to a method for sustaining islet cells or precursor cells in culture comprising culturing the cells in the presence of a GLP-1 agonist and a gastrin compound, composition, or conjugate of the invention in an amount sufficient to sustain the cells in culture. The cells may be sustained in culture for a significantly longer period of time compared with cells cultured in the absence of the compounds, composition or conjugate, or in the presence of a GLP-agonist or a gastrin compound alone. Culturing cells in the presence of a GLP-1 agonist and a gastrin compound or a composition or conjugate of the invention will be particularly useful in preparing and maintaining cells intended for transplantation.

In an aspect, the invention provides a method of treating a condition and/or disease comprising administering a GLP-1 agonist and a gastrin compound, a composition or conjugate of the invention with a plurality of cells to a subject in need thereof to thereby produce a beneficial effect, preferably a sustained beneficial effect.

A method for treating a subject with a condition and/or disease described herein comprises contacting ex vivo a plurality of cells with a GLP-1 agonist and a gastrin compound, or a composition or conjugate of the invention of the invention, optionally culturing the cells, and administering the cells to the subject in need thereof.

In embodiments of the aforementioned cell based therapeutic methods the cells are pancreatic ductal cells and the amount of compounds/composition/conjugate used in the method is generally effective to increase the amount of insulin secreting cells in the subject. The cells may be autologous (i.e. from the same subject), or may be from another individual of the same species, or from a different species.

The invention also contemplates a method for treating diabetes in a subject comprising transplanting a pancreatic islet preparation into the subject and administering a therapeutically effective amount of a GLP-1 agonist and a gastrin compound, or a composition or conjugate of the invention.

In the cell based methods of the invention the number of cells administered to an individual afflicted with a condition and/or disease will vary according to the severity of the condition and/or disease, the mode of administration, and/or the site of administration. Generally a therapeutically effective amount of cells is a safe and effective amount, and in particular an amount necessary to provide one or more beneficial effect, in particular a sustained beneficial effect, or a synergistic effect.

Cells can be administered to subjects using a variety of means apparent to those of skill in the art. Suitable methods include injection of the cells into a target site in a subject. Cells may be inserted into a delivery device to facilitate injection or implantation into the subjects. Examples of delivery devices include tubes, e.g., catheters, for injecting cells and fluids into the body of a subject Cells can be prepared for delivery in a variety of different forms. For example, the cells may be suspended in a solution or gel, or mixed with a pharmaceutically acceptable carrier, excipient, or diluent in which the cells remain viable. Pharmaceutically acceptable carriers, excipients, and diluents include saline, aqueous buffer solutions, solvents and/or dispersion media. The use of such carriers and diluents is well known in the art. The solution is generally sterile, and will often be isotonic. A solution of cells is preferably selected that is stable under the conditions of manufacture and storage and preserved against the contaminating action of microorganisms through the use of, for example, patabens, chlorobutanol, phenol, scorbic acid, thimerosal, and the like.

Modes of administration of cells include without limitation systemic intracardiac, intracoronary, intravenous, intradermal, or intra-arterial injection and injection directly into the tissue or organ at the intended site of activity, or in proximity to the site of activity. A cell preparation can be administered by any convenient route, for example by infusion or bolus injection and can be administered together with other biologically active agents. Administration in some aspects is preferably systemic. A cell preparation can be administered by any convenient route, for example by infusion or bolus injection and can be administered together with other biologically active agents.

Methods of the invention may further comprise measuring or monitoring one or more of the following markers: blood glucose, serum glucose, blood glycosylated haemoglobin, pancreatic beta cell mass, serum insulin, pancreatic insulin levels, morphometrically determined beta cell mass, amount of insulin secreting cells, and glucose responsiveness of insulin secreting cells.

The invention also contemplates the use of a composition comprising a combination of at least one GLP-1 agonist and at least one gastrin compound for the preparation of a medicament providing beneficial effects, preferably sustained beneficial effects in treating a condition and/or disease. In an aspect, the invention relates to the use of a therapeutically effective amount of at least one GLP-1 agonist, and at least one gastrin compound for preparation of a medicament for providing beneficial effects, preferably sustained beneficial effects, in treating a condition and/or disease. In an embodiment the invention provides the use of a GLP-1 agonist and a gastrin compound for the preparation of a medicament for increasing (preferably sustained increase) the number and/or size of beta cells in a subject after treatment. In another embodiment the invention provides the use of GLP-1 agonist and a gastrin compound for the preparation of a medicament for stimulation (preferably sustained stimulation) of beta cell proliferation after treatment. In a still further embodiment the invention provides the use of GLP-1 and Gastrin for the preparation of a medicament for treatment of Type I or Type II diabetes.

The invention additionally provides uses of a pharmaceutical composition and a conjugate of the invention in the preparation of medicaments for beneficial effects, preferably sustained beneficial effects, in the treatment of conditions and/or diseases.

Therapeutic efficacy and toxicity of compounds, compositions and conjugates of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

The compounds, compositions, medicaments, and conjugates of the present invention can be administered by any means that produce contact of the active agent(s) with the agent's sites of action in the body of a subject or patient. The active ingredients can be administered simultaneously or sequentially, and in any order at different points in time, to provide the desired beneficial effects. The compounds, conjugates and compositions can be formulated for sustained release, for delivery locally or systemically. It lies within the capability of a skilled physician or veterinarian to select a form and route of administration that optimizes the effects of the compositions, conjugates, and treatments of the present invention.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. The compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

A particular route of administration is parenteral administration, preferably peripheral parenteral administration. Parenteral administration is generally understood to refer to the injection of a dosage form into the body by a sterile syringe or some other mechanical device such as an infusion pump. For the purpose of the present invention parenteral routes include intravenous, intramuscular, subcutaneous, and intraperitoneal routes of administration. For parenteral administration, the compounds or conjugates described herein may be combined with distilled water at an appropriate pH.

The present invention includes combination treatments providing additive or synergistic activity, delivering an additive or synergistically effective amount, or an amount to provide a therapeutically effective amount of a GLP-1 agonist and a gastrin compound, or a conjugate or composition of the invention. Therefore, pharmaceutical compositions suitable for use in the present invention include compositions wherein the active ingredients are contained in a synergistically effective amount or a therapeutically effective amount.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect. The effective amount of a drug required to prevent, counter, or arrest progression of a condition can be readily determined by an ordinarily skilled physician or veterinarian.

A composition, medicament, or treatment of the invention may comprise a unit dosage of at least one GLP-1 agonist and a unit dosage of at least one gastrin compound. A “unit dosage” refers to a unitary i.e. a single dose which is capable of being administered to a patient, and which may be readily handled and packed, remaining as a physically and chemically stable unit dose comprising either the active agents as such or a mixture with one or more solid or liquid pharmaceutical excipients, carriers, or vehicles.

In an aspect, a pharmaceutical composition is provided comprising a therapeutically effective suboptimal dosage of a GLP-1 agonist and a gastrin compound that are more effective at decreasing or reducing glucose levels for a sustained period following treatment compared with a dosage of either a gastrin compound or GLP-1 agonist alone.

In another aspect, an improved pharmaceutical composition is provided comprising therapeutically effective suboptimal amounts of a GLP-1 agonist and a gastrin compound in a form for chronic or acute therapy of a condition and/or disease, in particular diabetes.

In an embodiment, the composition comprises a GLP-1 agonist and a gastrin compound in doses that are equal to or at least 1.1, 1.5, 2, 3, 4, 5, 6, 7, 8, 9, or 10 fold lower than the doses of each compound required to provide beneficial effects, preferably sustained beneficial effects, to treat a condition and/or disease.

In an aspect the invention provides a pharmaceutical composition comprising between 0.5 to 6000, 100-1500, 100-6000, 1000-6000, 2000-6000, and 3000-6000 micrograms GLP-1 agonist per single unit and 0.5 to 6000, 100-3000, 100-6000, 1000-6000, 2000-6000, and 3000-6000 micrograms gastrin compound per single unit.

In another aspect the invention provides a pharmaceutical composition comprising between 0.1 to 20, 0.1 to 30, 0.1 to 40, 0.1 to 50, and 0.1 to 60 micrograms/kg/day GLP-1 and 0.1 to 20, 0.1 to 30, 0.1 to 40, 0.1 to 50, and 0.1 to 60 micrograms/kg/day gastrin compound.

A composition or formulation of the invention may administered to a subject continuously for 2 weeks to 12 months, 2 weeks to 6 months, 2-16 weeks, 2 weeks to 12 weeks, and/or 2-8 weeks, or periodically.

In an embodiment, the ratio of GLP-1 agonist to gastrin compound in a composition of the invention is selected to augment the activity of the GLP-1 agonist and/or gastrin compound and to provide beneficial effects, preferably sustained beneficial effects.

A GLP-1 agonist and a gastrin compound may be in a ratio selected to augment the activity of one or both compounds to produce beneficial effects, in particular a sustained beneficial effect, and/or to produce an additive or synergistic effect. In embodiments, the ratio of a GLP-1 agonist to a gastrin compound may be from 1:1 to 1:110, 1:1 to 1:100, 1:1 to 1:75, 1:1 to 1:50, 1:1 to 1:25, 1:1 to 1:10, 1:1 to 1:5, and 1:1. In other particular embodiments, the ratio of a gastrin compound to a GLP-1 agonist may be from 1:1 to 1:110, 1:1 to 1:100, 1:1 to 1:75, 1:1 to 1:50, 1:1 to 1:25, 1:1 to 1:10, and 1:1 to 1:5.

A GLP-1 agonist may be used in combination with a gastrin compound at therapeutically effective weight ratios of between about 1:1 to 1:150, in particular 1:1 to 1:50. In another embodiment, a gastrin compound may be used in combination with a GLP-1 agonist at therapeutically effective weight ratios of between about 1:1 to 1:150, in particular 1:1 to 1:50.

The compositions of the present invention or fractions thereof typically comprise suitable pharmaceutical diluents, excipients, vehicles, or carriers selected based on the intended form of administration, and consistent with conventional pharmaceutical practices. The carriers, vehicles etc. may be adapted to provide an additive, synergistically effective or therapeutically effective amount of the active compounds.

Suitable pharmaceutical diluents, excipients, vehicles, and carriers are described in the standard text, Remington's Pharmaceutical Sciences, Mack Publishing Company. By way of example, for oral administration in the form of a capsule or tablet, the active components can be combined with an oral, non-toxic pharmaceutically acceptable inert carrier such as lactose, starch, sucrose, methyl cellulose, magnesium stearate, glucose, calcium, sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof.

In an aspect of the invention a pharmaceutical composition has a pH from about 7 to 10.

Formulations for parenteral administration of a composition of the invention may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

In an embodiment, a solid form pharmaceutical composition is provided (e.g. tablets, capsules, powdered, or pulverized form) comprising a crystalline or amorphous GLP-1 agonist and a crystalline or amorphous gastrin compound.

In another embodiment, the invention relates to a liquid drug formulation comprising pharmaceutically acceptable salts of a GLP-1 agonist and a gastrin compound, and to lyophilized drug formulations that can be reconstituted to provide suspensions that are stable and suitable for parenteral administration.

In a particular embodiment, the invention relates to an aqueous composition comprising pharmaceutically acceptable salts of a GLP-1 agonist and a gastrin compound, and a solvent system which effects solubilization. The invention also provides a drug comprising an aqueous formulation of pharmaceutically acceptable salts of a GLP-1 agonist and a gastrin compound with at least one solubilizer.

A composition of the invention may be sterilized by, for example, filtration through a bacteria retaining filter, addition of sterilizing agents to the composition, irradiation of the composition, or heating the composition. Alternatively, the compounds, conjugates, and compositions of the present invention may be provided as sterile solid preparations e.g. lyophilized powder, which are readily dissolved in sterile solvent immediately prior to use.

In addition to the formulations described herein, the compositions can also be formulated as a depot preparation. Such long acting formulations may be administered by implantation (for example, subcutaneously or intramuscularly) or by intramuscular injection. Thus, for example, the fractions may be formulated with suitable polymeric or hydrophobic materials (for example, as an emulsion in an acceptable oil), or ion exchange resins, or as sparingly soluble derivatives, for example, as a sparingly soluble salt.

The compositions of the invention and components thereof may comprise soluble polymers as targetable drug carriers.

After pharmaceutical compositions have been prepared, they can be placed in an appropriate container and labelled for treatment of an indicated condition. For administration of a composition of the invention, such labelling would include amount, frequency, and method of administration.

The present invention also includes methods of using the compositions of the invention in combination with one or more additional therapeutic agents including without limitation immunosuppressive agents, antiobesity agents, antidiabetic agents, appetite regulating drugs, antihypertensive agents, agents for the treatment and/or prevention of complications resulting from or associated with a condition and/or disease, in particular diabetes and obesity, anti-nausea, anti-headache medications, and general medications that treat or prevent side effects.

Since the present invention relates to a method of treatment comprising a combination of active agents which may be administered separately or as conjugates, the invention also provides a kit comprising a GLP-1 agonist and a gastrin compound, a pharmaceutical composition or conjugate in kit form. The invention also relates to a pharmaceutical kit comprising one bottle with a GLP-1 agonist and another bottle with a gastrin bottle in one box. A kit may comprise a package which houses a container which contains a conjugate or composition of the invention and also houses instructions for administering the conjugate or composition to a subject.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Effects of Gastrin in Combination with GLP-1 (Bachem, GLP-1(7-36) Amide, Human) in Acutely-Diabetic NOD Mice

This example shows methods and compositions for reversing diabetes in diabetic NOD mice by stimulating β-cell neogenesis in vivo following systemic treatment with GLP-1 and Gastrin. Female NOD mice ages 12-16 weeks were treated for 18 days only with vehicle (PBS), GLP-1 (300 μg/kg/day), or GLP-1 (300 μg/kg/day)+Gastrin (3 μg/kg/day), by injection intraperitoneally twice daily within 2 days after diabetes onset. Onset of diabetes was determined by fasting blood glucose (FBG) levels (9-15 mM compared with normal FBG <6.0 mM). The mice were monitored daily for urine glucose and weekly for FBG levels.

At the start of the treatments, fasting blood glucose levels ranged between 11-14 mM. After 18 days of treatments, FBG was 24±1 mM in vehicle-treated mice, 13±2 mM in mice treated with GLP-1 alone, and 6±1 mM in mice treated with the combination of GLP-1 and Gastrin (mean±SE, n=4 mice in treated groups; n=6 mice in the control group).

After 18 days of treatment therapy was stopped and FBG was monitored weekly for additional six weeks. One week following the completion of treatment, the FBG levels returned to normal in mice injected with the combination therapy, and they remained at such levels throughout the end of the study at six weeks post treatment. Comparatively, the untreated group of animals had to be sacrificed due to the severity of the disease after five weeks. Mice treated with GLP-1 alone showed transient improvement in FBG levels up to 2 weeks after the treatment was stopped, after which the fasting blood glucose levels progressively increased and were similar to the levels observed in non-treated vehicle group by the end of the study.

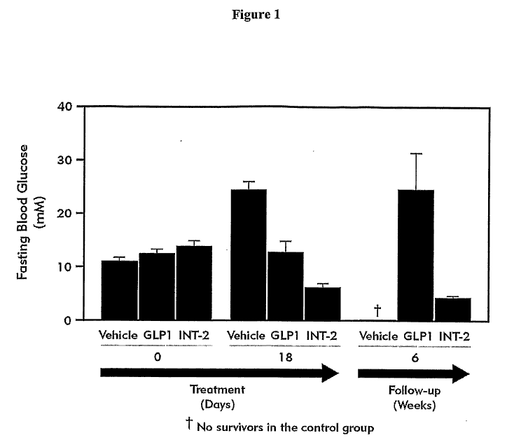

The results of the study are illustrated in FIG. 1.

These results show that a short course of a combined GLP-1 and gastrin treatment to diabetic NOD mice normalized hyperglycemia to effectively treat the diabetes, and it had a prolonged effect on fasting blood glucose levels indicating a stimulation of beta cell neogenesis and insulin production.

### Example 2

### Effects of Gastrin (G1) in Combination with GLP-1 in Acutely-Diabetic NOD Mice

### Objective:

NOD mice spontaneously develop insulin-dependent diabetes as a result of autoimmune destruction of pancreatic islet β-cells. This study was aimed to correct diabetes in NOD mice by regenerating islet β-cells using GLP-1 and gastrin (G1).

### Method:

Female NOD mice ages 12-16 weeks were treated for 18 days only, with vehicle (PBS) or with 300 μg/kg/day of GLP-1 in combination with 3 μg/kg/day of Gastrin (G1) by intraperitoneal injection (i.p.) Animals were injected for 18 days, twice daily, within 2 to 5 days after diabetes onset. The fasting blood glucose (FBG) levels were 9-15 mM at diabetes onset (normal FBG<6.0 mM). The mice were monitored daily for urine glucose levels and weekly for FBG levels during the treatment and for an additional 6 weeks after the treatment was stopped. The pancreatic insulin levels were determined in each group as well as histological analysis of the pancreatic tissue was performed. Pancreatic tissues were fixed and stained for insulin producing cells. The beta cell mass was determined by morphometric analysis.

### Results:

After 18 days of daily treatments, in animals treated with the combination of 30 μg/kg/day of GLP-1 and 3 μg/kg/day of G1, fasting blood glucose was 6.1±0.7 mM, whereas the fasting blood glucose was 24.4±1.5 mM in vehicle treated group. In comparison, the animals treated with GLP-1 alone had fasting blood glucose levels of 12.5±2.2 mM. These data indicate that GLP-1 and Gastrin combination treatment was more effective than GLP-1 alone in controlling glucose levels in NOD mice.

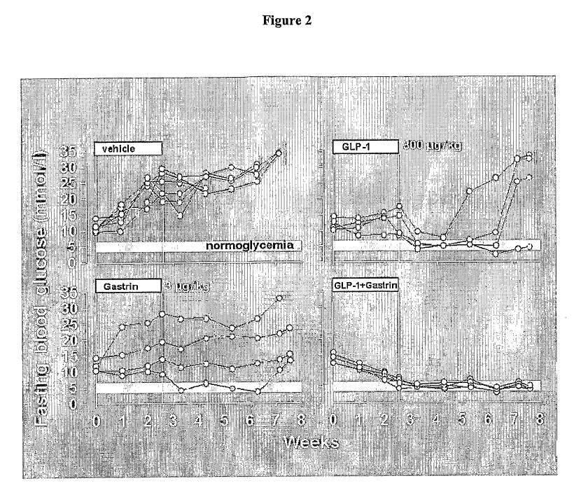

All treatments were stopped after 18 days and FBG was monitored weekly for an additional six weeks. One week following the completion of treatment FBG levels were normal (below 6 mM) in mice injected with the combination therapy and remained at such levels throughout the end of the study with FBG of 4.3±0.2 mM at six weeks post treatment. Comparatively, the GLP-1 treated animals reached maximal blood glucose levels (over 30 mM) and were suffering from diabetic complications. The untreated group of animals had to be sacrificed due to the severity of the disease after five weeks post treatment (FIG. 1 and FIG. 2).

These data indicate that the GLP-1 and gastrin combination was effective in restoring normal blood glucose levels even after 6 weeks post-treatment, whereas GLP-1 treated animals developed severe hyperglycemia.

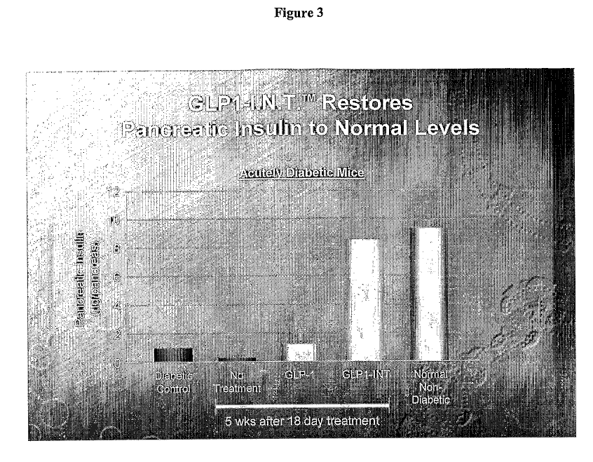

FIG. 3 demonstrates that non-diabetic animals have approximately 10 μg of insulin per pancreas, whereas acutely diabetic animals with elevated glucose levels have 0.5 to 1.0 μg of pancreatic insulin. These data show that NOD mice require less than 10% of their pancreatic insulin to regulate glucose levels. Five weeks following the onset of diabetes the untreated animals had minimal levels of pancreatic insulin, and at this stage the animals had glucose levels of 30-32 mM and suffer from diabetic complications. The GLP-1 treated group had pancreatic insulin levels of 1.0 to 1.5 μg, which is higher than untreated animals suggesting that GLP-1 stimulates some islet cell regeneration in the NOD mouse model.

Strikingly, the GLP-1 and Gastrin treated animals had over 8 μg per pancreas which is not only significantly higher than GLP-1 but over 80% of normal non-diabetic pancreatic insulin levels. These studies show that a GLP-1 and Gastrin combination treatment is very robust in stimulating islet cell regeneration that is capable of reversing disease for long periods of time post treatment.

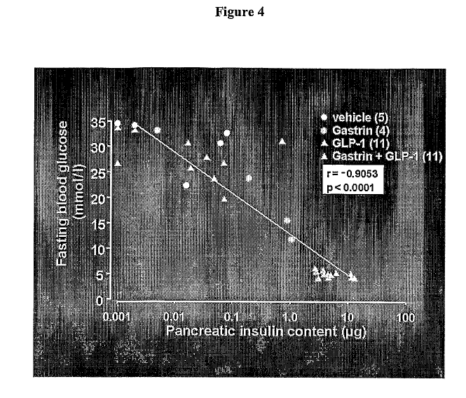

GLP-1 and Gastrin were able to restore pancreatic insulin content from the low levels measured after diabetes onset and before treatment to a level similar to that measured in normoglycemic mice. Correction of hyperglycemia in NOD mice was significantly correlated with the increase in pancreatic insulin content (r=0.90), as presented in FIG. 4.

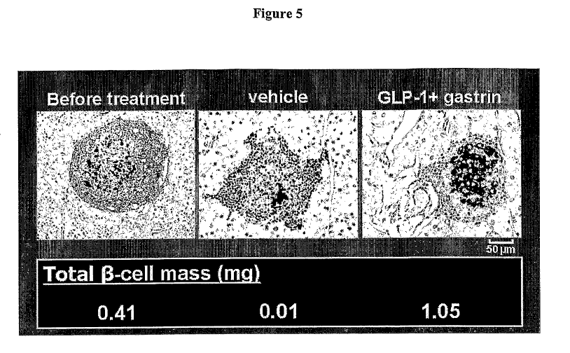

FIG. 5 shows that insulin stained cells (in dark brown), are few in acutely diabetic NOD mice before treatment. The number of these islet cells decrease further in the untreated group over time. Histologic examination revealed large, intensely insulin-stained islets adjacent to pancreatic ducts and surrounded but not invaded by mononuclear leukocytes in GLP-1 and Gastrin treated mice.

The beta cell mass decreased from 0.41 mg to 0.01 mg during the course of the experiment (8 weeks), whereas the beta cell mass increased to 1.05 mg in the group of animals treated with GLP-1 and Gastrin. The beta cell mass in non-diabetic animals has been reported to be in the 1.0-1.5 mg range. The GLP-1 and Gastrin treatment significantly increase beta cell mass in NOD mice to near normal levels, even when examined 6 weeks post treatment.

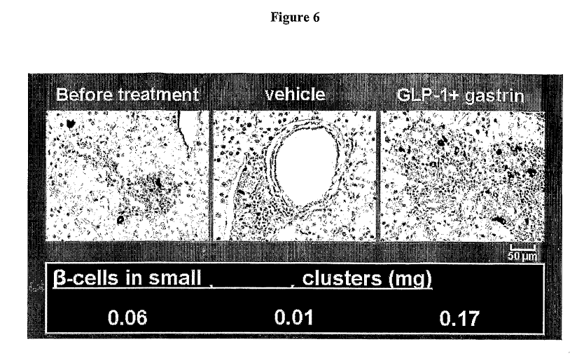

FIG. 6 demonstrates staining of islet cells from the pancreatic duct in NOD mice treated with vehicle and GLP-1 and Gastrin. The data demonstrate that beta cell mass of islet cell cluster in the pancreatic ducts decrease from 0.06 to 0.01 during the course of the experiment (8 weeks), whereas these clusters of beta cell mass increased to 0.17 mg in GLP-1 and Gastrin treated NOD mice. These data indicate that GLP-1 and Gastrin treatment induces islet neogenesis in NOD mice involving islet cell precursors in the pancreatic duct.

In summary, these studies show that GLP-1 and Gastrin treatment induce islet cell regeneration in the NOD mouse model sufficiently to outbalance the destruction that may be ongoing in these disease models, resulting in net accumulation of islet cells in the pancreas.

### Conclusion:

A short course of GLP-1 and Gastrin treatment of diabetic NOD mice normalizes hyperglycemia and has a prolonged effect on fasting blood glucose levels for periods of at least 6 weeks post treatment. In addition, the data show that GLP-1 and Gastrin is capable of stimulating pancreatic insulin levels that approximate 80-90% of normal levels, whereas GLP-1 alone has a modest effect only.

Furthermore, histological analysis of the pancreas shows that the islet cells appear normal and with large numbers of insulin producing cells, despite being surrounded by inflammatory cells. Morphometric analysis of the pancreas shows that GLP-1 and Gastrin treatment increases beta cell mass in the pancreas, and shows signs of inducing neogenesis by increasing beta cell mass in pancreatic ducts. GLP-1 and Gastrin treatment is a potent inducer of islet cell regeneration that is capable of restoring normal glucose and pancreatic insulin levels in the NOD mouse model.

### Example 3

### Modified Gastrin Compounds/Conjugates of PCT/CA03/01778 in Combination with GLP-1 in Preventing Diabetes Progression in NOD Mice with Recent Onset Diabetes

The effect of treatment by a combination of GLP-1 and unmodified gastrin and GLP-1 and modified gastrin compounds/conjugates will be examined in NOD mice with recent onset diabetes, to determine whether administration of both GLP-1 and gastrin prevents severe hyperglycemia as well as increase pancreatic insulin content in NOD mice with recent-onset diabetes. The GLP-1 to be used is the GLP-1 biologically active fragment of human/mouse GLP-1 (having residues at positions 7-36 compared to the precursor from which the fragment is processed; obtained from Bachem H6795). Gastrin compounds/conjugates to be used are as follows: Compound B—gastrin as synthetic human gastrin I having 17 amino acid residues with a Leu residue at amino acid position 15, Compound E—gastrin as synthetic human gastrin I having 2-17 amino acid residues, Compound Q gastrin as synthetic human gastrin I having 2-17 amino acid residues with a HSA polymer linked via (GA)₅ (i.e. Gly-Ala-Gly-Ala-Gly-Ala-Gly-Ala-Gly-Ala).

Non-obese diabetic (NOD) female mice, ages 12-14 weeks, will be monitored for development of onset of diabetes (fasting blood glucose >8.0 to 15 mmol/l), and within 48 hours after onset of symptoms, four groups of mice will each be treated as follows: one group will be treated with vehicle only, and the other group will be administered 100 μg/kg/day of GLP-1, and the remaining groups will be treated with a combination of GLP-1 (100 μg/kg/day) and gastrin compound (3 μg/kg/day gastrin equivalent), each treatment administered via the intraperitoneal route daily.

Therapy will be administered for 14 days to 18 days. Animals will be monitored weekly for fasting blood glucose (FBG) levels. FBG levels will be measured at about 12 hours after food has been withdrawn, and 24 hours after the last peptide or vehicle injection. Upon cessation of therapy, all mice will be monitored for FBG levels for the next 4 weeks (weeks 2-6) so as to determine whether prevention of hyperglycemia persisted after termination of therapeutic treatment. At 14 days to 18 days treatment will be stopped.

The protocol includes sampling of these mice for data again at 6 weeks, and blood collecting blood for assay of FBG and plasma C-peptide, and sacrificing the mice for pancreatic insulin determinations and scoring of islet inflammation (insulitis). From the outset of treatment, mice will neither receive insulin-replacement treatment nor immunosuppression. The following parameters will be assessed: survival rates, pancreatic insulin levels, presence of islet inflammation and fasting blood glucose levels.

GLP-1 in combination with a modified gastrin compounds/conjugates (Compound B or Q) with longer half lives may provide enhanced reduction of blood glucose levels in diabetic animals.

### Example 4

Using standard Fmoc synthesis, two different “reactive” gastrin compounds will be produced: Compound A is a Modified gastrin-17 peptide that has an additional cysteine at the N-terminal end; Compound B is a Modified gastrin-17 peptide that has an additional 10 amino acids of alternating glycine and alanine (5 amino acids each) as a spacer region with an additional cysteine at the N-terminal end.

Non-obese diabetic (NOD) female mice will be monitored for diabetes development (determined to be a fasting blood glucose, FBG level of greater than 6.6 mmol/l), and upon onset of diabetes, will be divided into four groups. Mice will be treated with either vehicle as a control; or with gastrin-17, with Compound A, or with Compound B (same molar concentration of active ingredient, i.e. gastrin, is to be used for all three gastrin treated groups), administered via intraperitoneal injection (i.p.) once daily for 14 days.

Fasting blood glucose (FBG) levels and pancreatic insulin levels will be measured determined. In addition, the serum half-life of gastrin will be measured as well as the circulating serum levels of gastrin

It is anticipated that of the three gastrin-treated groups, both groups of NOD mice that are treated either with Compound A or Compound B will maintain higher circulating levels of serum gastrin. In addition, the half-life of gastrin measured will be longer in mice treated with either Compound A or B as compared to the unmodified gastrin.

In addition, it is also anticipated that as compared to the vehicle treated control group which records increasingly high FBG levels, all three treated groups of animals will have decreased FBG levels. Animals treated with either Compound A or Compound B may have even lower FBG levels as well as increased pancreatic insulin levels compared to animals treated with unmodified gastrin.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant invention.

TABLE 1

| GLP-1 agonist | Source |
|---|---|
| DAC:GLP-1 | Conjuchem |
| Long-lasting synthetic glucagons-like peptide | Conjuchem |
| Long-lasting insulinotropic peptides | Conjuchem |
| AC2592 | Amylin Pharmaceuticals/Restoragen |
| AC2993 - Exenatide | Amylin Pharmaceuticals |
| Exendin-4 | Eli Lilly, Alkermes, Amylin |
| NN2211 - GLP-1 (Liraglutide) | Novo Nordisk |
| ThGLP-1 | Theratechnologies |
| ZP10 | Zealand Pharma/Aventis |
| Albumin:GLP-1 fusion peptide | Human Genome Sciences |
| BIM 51077 | Roche/Ipsen |
| N-terminally truncated GLP-1 derivatives & analogs | Novo Nordisk |
| (lipophilic substituent attached) | PCT/DK99/00081 |
| Derivatives of GLP-1 analogs with a lipophilic | Novo Nordisk |
| substituent | PCT/DK99/00082 |
| | U.S. Pat. No. 6,458,924 |
| N-terminally modified GLP-1 derivatives & analogs | Novo Nordisk |
| with lipophilic substituent attached and protracted | PCT/DK99/00085 |
| profile of action (N-terminal end has a substituent | |
| comprising an optionally substituted 5- or 6-membered | |
| ring system) | |
| Derivatives of GLP-1 analogs with a lipophilic | Novo Nordisk |
| substituent (protracted profile of action) | WO 98/08871 |
| GLP-1 fragment as insulinotropic hormone | The General Hospital Corporation |
| | WO 87/06941 |
| GLP-1 derivatives with insulinotropic activity | The General Hospital Corporation |
| | WO 90/11296 |
| GLP-1 analogs exhibiting enhanced stability or an | Buckley et al. |
| enhanced capacity to stimulate insulin production | WO 91/11457 |
| GLP-1 analogs and derivatives (stimulate the secretion | Eli Lilly & Co. |
| or biosynthesis of insulin in poorly functioning beta | EP 0708179-A2 |
| cells) | |
| N-terminal truncated GLP-1 and analogs (promote | Eli Lilly & Co. |
| glucose uptake by cells but do not stimulate insulin | EP 0699686 -A2 |
| expression or secretion) | |
| GLP-1 analogs or derivatives for increasing the number | Novo Nordisk |
| and/or the size of beta cells and for stimulating beta cell | US 2003/0224983 |
| proliferation | |
| GLP-1 derivatives with a lipophilic substituent and | Novo Nordisk |
| protracted profile of action | U.S. Pat. No. 6,268,343 |
| Pharmaceutical formulations of GLP-1 agonists | Novo Nordisk |
| | US 20030119734 A1 |
| GLP-1 amide, fragment, analogue or derivative | Novo Nordisk |
| | US 20030083259 A1 |
| GLP-1 compositions having protracted action | Novo Nordisk |
| | US 20010006943 A1 |
| GLP-1 & gastrin | Transition Therapeutics |
| | PCT/CA03/ |
| Gastrin formulations | Transition Therapeutics |
| | PCT/CA03/ |
| Derivatives of GLP-1 analogs with a lipophilic | Novo Nordisk |
| substituent (protracted profile of action) | WO 99/43706 |
| GLP-1 and exendin derivatives with just one lipophilic | Novo Nordisk |
| substituent attached to the C-terminal amino acid | WO 99/43708 |
| residue | |
| Modified exendins and agonists linked to one or more | Amylin Pharmaceuticals |
| polyethylene glycol polymers | WO 00/66629 |
| Ecarin, a procoagulant protein from Echis carinatus | Cohesion Technologies |
| venom | WO 01/04146 |
| Modified Fragments of GLP-1, exendin 3 and exendin | Conjuchem, Inc. |
| 4 | U.S. Pat. No. 6,514,500 |
| GLP-1 analogs | Novo Nordisk A/S |
| | U.S. Pat. No. 6,451,974 |
| GLP-1 analogs, derivatives and active peptides | Eli Lilly and Company |
| | 6,191,102 |
| GLP-1 Fragments | The General Hospital Corporation |
| | 6,162,907 |
| GLP-1 molecules associated with a divalent metal | Eli Lilly and Company |
| cation | 6,133,235 |
| | 5,977,071 |
| Buccal delivery systems with GLP-1 | Theratech, Inc. |
| | 5,863,555 |
| GLP-1 Analogs | Eli Lilly and Company |
| | 5,981,488 |
| GLP-1 mimics | Bristol-Myers Squibb Company |
| | WO 03/033671 |
| Long lasting GLP-1 | Conjuchem, Inc. |
| | U.S. Pat. No. 6,593,295 |
| | U.S. Pat. No. 6,514,500 |
| | U.S. Pat. No. 6,329,336 |
| Precursor GLP-1 | Genzyme Corporation |
| | WO 03/014318 |
| GLP-1 complexes | Eli Lilly and Company |
| | 6,358,924 |
| Modified peptides | Theratechnologies Inc. |
| | WO 02/10195 |
| GLP-1 and related molecules | Zealand Pharma A/S |
| | WO 2004/005342 |

1. A pharmaceutical composition comprising a GLP-1 agonist and a gastrin compound that provides beneficial effects relative to each compound alone, and a pharmaceutically acceptable carrier, excipient, or vehicle.

2-5. (canceled)

6. A pharmaceutical composition as claimed in claim 1 wherein the ratio of a GLP-1 agonist to a gastrin compound is from about 1:1 to 1:110, 1:1 to 1:100, 1:1 to 1:75, 1:1 to 1:50, 1:1 to 1:25, 1:1 to 1:10, 1:1 to 1:5, and 1:1.

7-8. (canceled)

9. A pharmaceutical composition as claimed in claim 1 wherein the GLP-1 agonist and the gastrin compound are present in doses that are at least about 1.1 to 1.4, 1.5, 2, 3, 4, 5, 6, 7, 8, 9, or 10 fold lower than the doses of each compound alone required to treat a condition and/or disease.

10. A pharmaceutical composition as claimed in claim 1 comprising an additive amount of the GLP-1 agonist and the gastrin compound in a pharmaceutically acceptable excipient, carrier, or vehicle.

11. A pharmaceutical composition as claimed in claim 1 comprising a synergistically effective amount of the GLP-1 agonist and the gastrin compound in a pharmaceutically acceptable excipient, carrier, or vehicle.

12. A pharmaceutical composition as claimed in claim 1 comprising between 0.1 to 20, 0.1 to 30, 0.1 to 40, 0.1 to 50, and 0.1 to 60 micrograms/kg/day GLP-1 agonist and 0.1 to 20, 0.1 to 30, 0.1 to 40, 0.1 to 50, and 0.1 to 60 micrograms/kg/day gastrin compound.

13-18. (canceled)

19. A pharmaceutical composition as claimed in claim 1 wherein the beneficial effect is a decrease in blood glucose levels for a period of at least 2, 4, 6, 8, or 10 weeks, 2 to 4 weeks, 2 to 6 weeks, 2 to 8 weeks, 2 to 12 weeks, 2 to 24 weeks, 2 weeks to 12 months, and 2 weeks to 18 months following treatment.

20. A pharmaceutical composition as claimed in claim 1 wherein the GLP-1 agonist is a GLP-1(1-37), GLP-1(7-36) amide, fragments, analogues, and derivatives thereof, and active metabolites and prodrugs of GLP-1.

21-23. (canceled)

24. A pharmaceutical composition as claimed in claim 1 wherein the gastrin compound is gastrin 71 [SEQ ID NO. 15], gastrin 52 [SEQ ID NO. 16], gastrin 34 (big gastrin) [SEQ ID NO. 11 or 12], gastrin 17 (little gastrin) [SEQ ID NO. 13 or 14], gastrin 14 [SEQ ID NO. 17], gastrin 8, gastrin 6 [SEQ ID NO.18 or 19], pentagastrin, and tetragastrin, or a fragment, analog, or derivative thereof.

25-26. (canceled)

27. A pharmaceutical composition of claim 1 wherein the GLP-1 agonist is Arg34Lys26(Ne(g-Glu(Na-hexadecanoyl)))-GLP-1(7-37) and the gastrin compound is 15Leu gastrin 17 [SEQ ID NO. 14].

28. (canceled)

29. A method for preparing a stable pharmaceutical composition of a GLP-1 agonist comprising mixing a GlP-1 agonist, a gastrin compound, and a pharmaceutically acceptable carrier, excipient, or vehicle effective to physically stabilize the GLP-1 agonist and adapted to provide beneficial effects.

30-33. (canceled)

34. A method of treatment comprising administering to a subject a therapeutically effective amount of a composition according to claim 1 or at least one GLP-1 agonist in combination with administration of at least one gastrin compound which upon administration to a subject with symptoms of diabetes provides sustained beneficial effects.

35. (canceled)

36. A method as claimed in claim 34 wherein therapeutically effective amounts of the GLP-1 agonist and the gastrin compound are combined prior to administration to the subject.

37. A method as claimed in claim 34 wherein therapeutically effective amounts of the GLP-1 agonist and the gastrin compound are administered to the subject sequentially.

38-39. (canceled)

40. A method of treating diabetes comprising administering a composition of claim 1 with a plurality of cells to a subject in need thereof to thereby produce beneficial effects.

41-51. (canceled)

52. A pharmaceutical composition according to claim 24 wherein the GLP-1 agonist is an exendin or analog, derivative or fragment thereof.

53. A method according to claim 34 wherein the GLP-1 agonist is an exendin or analog, derivative or fragment thereof.

54. A method according to claim 54 wherein the GLP-1 agonist is exendin-3 or exendin-4.

55. A method according to claim 54 wherein the GLP-1 agonist is exenatide.


SEQUENCE LISTING (27 sequences)

SEQ ID NO: 1 - protein, 37 aa, Artificial Sequence
  Chemically Synthesized
HDEFERHAEGTFTSDVSSYLEGQAAKEFIAWLVKGRG

SEQ ID NO: 2 - protein, 36 aa, Artificial Sequence
  Chemically Synthesized
HDEFERHAEGTFTSDVSSYLEGQAAKEFIAWLVKGR

SEQ ID NO: 3 - protein, 36 aa, Artificial Sequence; identical to SEQ ID NO: 2
  Chemically Synthesized

SEQ ID NO: 4 - protein, 31 aa, Artificial Sequence
  Chemically Synthesized
HAEGTFTSDVSSYLEGQAAKEFIAWLVKGRG

SEQ ID NO: 5 - protein, 30 aa, Artificial Sequence
  Chemically Synthesized
HAEGTFTSDVSSYLEGQAAKEFIAWLVKGR

SEQ ID NO: 6 - protein, 30 aa, Artificial Sequence; identical to SEQ ID NO: 5
  Chemically Synthesized

SEQ ID NO: 7 - protein, 39 aa, Heloderma horridum
HSDGTFTSDLSKQMEEEAVRLFIEWLKNGGPSSGAPPPS

SEQ ID NO: 8 - protein, 39 aa, Heloderma suspectum
HGEGTFTSDLSKQMEEEAVRLFIEWLKNGGPSSGAPPPS

SEQ ID NO: 9 - protein, 31 aa, Artificial Sequence
  Chemically Synthesized
HGEGTFTSDLSKQMEEAVRLFIEWLKNGGPY

SEQ ID NO: 10 - protein, 31 aa, Artificial Sequence
  Chemically Synthesized
DLSKQMEEEAVRLFIEWLKNGGPSSGAPPPS

SEQ ID NO: 11 - protein, 32 aa, Artificial Sequence
  Chemically Synthesized
XLGPQGPPHLVADPSKKQGPWLEEEEEAYGWM

SEQ ID NO: 12 - protein, 32 aa, Artificial Sequence
  Chemically Synthesized
XLGPQGPPHLVADPSKKQGPWLEEEEEAYGWL

SEQ ID NO: 13 - protein, 16 aa, Artificial Sequence
  Chemically Synthesized
XGPWLEEEEEAYGWMD

SEQ ID NO: 14 - protein, 16 aa, Artificial Sequence
  Chemically Synthesized
XGPWLEEEEEAYGWLD

SEQ ID NO: 15 - protein, 101 aa, Artificial Sequence
  Chemically Synthesized
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN

SEQ ID NO: 16 - protein, 52 aa, Artificial Sequence
  Chemically Synthesized
DLELPWLEQQGPASHHRRQLGPQGPPHLVADPSKKQGPWLEEEEEAYGWMDF

SEQ ID NO: 17 - protein, 14 aa, Artificial Sequence
  Chemically Synthesized
WLEEEEEAYGWMDF

SEQ ID NO: 18 - protein, 6 aa, Artificial Sequence
  Chemically Synthesized
YGWMDF

SEQ ID NO: 19 - protein, 6 aa, Artificial Sequence
  Chemically Synthesized
YGWLDF

SEQ ID NO: 20 - protein, 31 aa, Artificial Sequence
  Chemically Synthesized
HGEGTFTSDLSKQMEEEAVRLFIEWLKNGGX

SEQ ID NO: 21 - protein, 40 aa, Artificial Sequence
  Chemically Synthesized
HXXGTFITSDLSKQMEEEAVRLFIEWLKNGGPSSGAPPPS

SEQ ID NO: 22 - protein, 52 aa, Artificial Sequence
  Chemically Synthesized
HGEGTFTSDLSKQMEEEAVRLFIEWLKNGGPSSGAPPSKKKKKKSSGAPPPS

SEQ ID NO: 23 - protein, 6 aa, Artificial Sequence
  Chemically Synthesized
YGWMDF

SEQ ID NO: 24 - protein, 6 aa, Artificial Sequence
  Chemically Synthesized
YGWLDF

SEQ ID NO: 25 - protein, 10 aa, Artificial Sequence
  Chemically Synthesized
GAGAGAGAGA

SEQ ID NO: 26 - protein, 4 aa, Artificial Sequence
  Chemically Synthesized
WMDF

SEQ ID NO: 27 - protein, 4 aa, Artificial Sequence
  Chemically Synthesized
WLDF